(19) United States
(12) Patent Application Publication
(10) Pub. No.: US 2015/0104484 A1
(43) Pub. Date: Apr. 16, 2015

(54) STORED STRAIN POLYELECTROLYTE COMPLEXES AND METHODS OF FORMING

(71) Applicant: THE FLORIDA STATE UNIVERSITY RESEARCH FOUNDATION, INC., Tallahassee, FL (US)

(72) Inventors: Joseph B. Schlenoff, Tallahassee, FL (US); Qifeng Wang, Tallahassee, FL (US)

(21) Appl. No.: 14/492,144

(22) Filed: Sep. 22, 2014

Related U.S. Application Data
(60) Provisional application No. 61/890,548, filed on Oct. 14, 2013.

(51) Int. Cl. A61K 47/48; A61K 45/06

(52) U.S. Cl. CPC A61K 47/48184; A61K 45/06
USPC 424/400; 514/772.5; 514/7.6; 514/11.3

(57) ABSTRACT

The present disclosure is directed to articles comprising a polyelectrolyte complex which stores mechanical strain and methods of forming articles comprising a polyelectrolyte complex which stores mechanical strain.

### CROSS REFERENCE TO RELATED APPLICATIONS

This application claims priority from U.S. Provisional Application Ser. No. 61/890,548 filed Oct. 14, 2013, the disclosure of which is incorporated herein as if set forth in its entirety.

### STATEMENT REGARDING FEDERALLY SPONSORED RESEARCH OR DEVELOPMENT

This invention was made with government support under DMR 1207188 awarded by the National Science Foundation. The government has certain rights in the invention.

### FIELD OF THE INVENTION

The present invention relates to articles comprising a polyelectrolyte complex which stores mechanical strain and methods of forming articles comprising a polyelectrolyte complex which stores mechanical strain.

### BACKGROUND OF THE INVENTION

Hydrogels comprise water and polymers and are useful for medical and pharmaceutical applications (e.g. see Peppas, N. A.; Editor, Hydrogels in Medicine and Pharmacy, Vol. 3: Properties and Applications. 1987; p 195 pp.). Hydrogels are usually held together via physical or chemical crosslinks, otherwise the polymers of which they are comprised would dissolve in the solvent (water). Polyelectrolyte complexes are interpenetrating complexes of one or more predominantly positive polyelectrolytes and one or more predominantly negative polyelectrolytes. The opposite charges on the polymers form ion pairs between chains, holding the chains together. This ion pairing is a type of physical crosslinking. Polyelectrolyte complexes in contact with aqueous solutions can be considered hydrogels with high crosslinking density.

There is a need to prepare articles with dimensions in the millimeter to centimeter to meter scale to provide materials and shapes for biomedical and engineering applications. Polyelectrolyte complexes are prepared in a straightforward manner by mixing solutions of positive and negative polyelectrolytes. However, the resulting precipitate is gelatinous and difficult to process. The dried complexes, for example, are generally infusible and therefore cannot be injection molded or reformed into articles under elevated temperatures. Michaels (U.S. Pat. No. 3,324,068) has disclosed the used of non-volatile plasticizers such as nonvolatile acids, organic oxysulfur compounds and organic oxyphosphorous compounds to decrease the brittleness of polyelectrolyte complexes when they are dried. U.S. Pat. No. 3,546,142 describes a method for creating solutions of polyelectrolyte complexes using aggressive ternary solvents which are mixtures of salt, water and organic solvent. Said solutions of complexes may be cast into films by evaporating the solvent. Mani et al. (U.S. Pat. No. 4,539,373) point out that the solid complexes “are not thermoplastic, i.e. they are not moldable or extrudable, so they must be handled as solutions.” Mani et al. disclose a polyelectrolyte complex comprising nonionic thermoplastic repeat units which can be thermally molded.

U.S. Pat. Nos. 8,114,918; 8,222,306; 8,283,030; 8,314,158; and 8,372,891 and U.S. Pat. Pub. No. 20090162640 which are incorporated fully by reference, disclose how fully hydrated (i.e. complexes in contact with water) polyelectrolyte complexes may be reformed into shapes without raising the temperature, without the addition of organic solvent, and without the need for dissolution, if they are doped with salt ions to a sufficient extent.

While the polyelectrolyte complex articles resulting from methods described in U.S. Pat. Nos. 8,114,918; 8,222,306; 8,283,030; 8,314,158; and 8,372,891 are tough when wet (hydrated), when they are dry the articles are brittle and fragile. In fact, the brittleness of dried polyelectrolyte complexes, prepared by any and all means, is widely known and is often cited as a reason they are not in widespread use. There is a need, therefore, to produce tougher dry formats of polyelectrolyte complex.

### SUMMARY OF THE INVENTION

Among the various aspects of the present invention may be noted an article comprising an interpenetrating network of at least one predominantly positively charged polyelectrolyte and at least one predominantly negatively charged positive polyelectrolyte. The polyelectrolyte in said material contains stored strain in one or two dimensions.

In one embodiment, the present invention is directed to an article comprising a polyelectrolyte complex comprising an interpenetrating network of at least one predominantly positively charged polyelectrolyte polymer and at least one predominantly negatively charged polyelectrolyte polymer, the polyelectrolyte complex further comprising stored strain with a stored strain factor of at least 2.

In one embodiment, the present invention is directed to a method of releasing stored strain from the article comprising polyelectrolyte complex further comprising stored strain with a stored strain factor of at least 2. The method comprises contacting the polyelectrolyte complex having stored strain with water to thereby hydrate the polyelectrolyte complex; and exposing the hydrated polyelectrolyte complex to a stimulus sufficient to release stored strain from the polyelectrolyte complex, said stimulus being selected from the group consisting of salt concentration increase, temperature increase, and pH change.

In one embodiment, the present invention is directed to a method of forming an article comprising a polyelectrolyte complex comprising an interpenetrating network of at least one predominantly positively charged polyelectrolyte polymer and at least one predominantly negatively charged polyelectrolyte polymer, the polyelectrolyte complex further comprising stored strain with a stored strain factor of at least 2. The method comprises contacting a polyelectrolyte complex comprising an interpenetrating network of at least one predominantly positively charged polyelectrolyte polymer and at least one predominantly negatively charged polyelectrolyte polymer with water to thereby hydrate the polyelectrolyte complex; and applying an external stress to the hydrated polyelectrolyte complex, the external stress sufficient to increase at least one dimension of the hydrated polyelectrolyte complex.

In one embodiment of the article, stored strain is created by stressing the polyelectrolyte complex by deforming the fully hydrated complex without the presence of a low molecular weight salt.

In another embodiment the article is formed by forcing a material comprising a hydrated salt-free blend of interpenetrating positive and negative polyelectrolyte into a mold under pressure and said article adopts and maintains the contours of the mold following release from the mold.

In another embodiment the article is formed by forcing a material comprising a hydrated salt-free blend of interpenetrating positive and negative polyelectrolyte through an orifice, said orifice defining the cross section of the article as it passes through the orifice.

Other objects and features will be in part apparent and in part pointed out hereinafter.

### BRIEF DESCRIPTION OF THE DRAWINGS

FIGS. 1A and 1B are optical autofluorescence microscopy images of 10 μm thick slices of polyelectrolyte complexes precipitated in 0.25 M NaCl, and centrifuged. FIG. 1A depicts the polyelectrolyte complex as extruded, and FIG. 1B depicts the polyelectrolyte complex soaked in DI water. 450-490 nm excitation and 500-550 nm emission filter cube. Scale bar: 100 μm.

FIG. 2 is a graph depicting room temperature water content vs. salt concentration for PSS/PDADMA Polyelectrolyte complexes after hydration for 2 days in salt solutions. The data is shown for polyelectrolyte complex extruded (), double extruded (⋄), and triple extruded (▴).

FIG. 3 is a graph depicting doping level, y, in PSS/PDADMA extruded polyelectrolyte complex (exPEC) versus salt activity for NaF (); NaCH₃COO (⋄); NaClO₃ (♦); NaCl (▪); NaNO₃ (Δ) NaBr (∘); NaI (♦); NaClO₄ (x); and NaSCN (□). Room temperature.

FIG. 4 is a graph depicting stress relaxation of extruded PEC doped in different NaCl concentrations and strained rapidly to 2%: 0.1M (a), 0.25 M (b), 0.5 M (c), 0.75 M (d), 1.0 M (e), and 1.25 M (f) NaCl.

FIG. 5 is a graph depicting equilibrium modulus at different salt solutions for PSS/PDADMA samples extruded (), double extruded (⋄), and triple extruded (▴) at strain of 2% and speed of 10 mm/min. The points (x) are the modulus for PEMU of PDADMA/PSS recorded with 70 mS relaxation time.

FIGS. 6A through 6D are images of extruded polyelectrolyte complex with salt.

FIG. 7 is a graph depicting Strain to Break test for stored strain and annealed PEC fibers. Stretching speed: 10 mm min⁻¹ (50% strain min⁻¹).

FIG. 8 are photographs of stored strain fibers in a tight knot (FIG. 8A) and the maximum degree (˜58°) the annealed sample can be bent (FIG. 8b).

FIG. 9 depicts length change (contraction) of stored strain polyelectrolyte complex samples in NaCl solutions (0-2.0 M) with time (FIG. 9A). And the minimum length in solutions of different [NaCl] (FIG. 9B).

FIG. 10 is a graph depicting release of stored strain polyelectrolyte complex by 90° C. water. The stored strain ratio is about 5.

### DESCRIPTION OF THE EMBODIMENT(S) OF THE INVENTION

One aspect of the invention is an article comprising a polymer, in particular, a polymer known as a “polyelectrolyte” that comprises multiple electrolytic repeat units that dissociate in solutions, making the polymer charged. The article of the present invention comprises a polyelectrolyte complex, that is, an intermolecular blend of a predominantly positively-charged polyelectrolyte and a predominantly negatively-charged polyelectrolyte. The polyelectrolyte complex is preferably compacted, such as by centrifugation or pressure, in a manner that increases the density of the polyelectrolyte complex to a value substantially greater than that which may be obtained following precipitation. Moreover, the article may be reformed or reshaped to have dimensions typically on the order of millimeters to centimeters, which is also substantially greater than that achievable by conventional multilayering (as described for example in Science, 277 p 1232-1237 (1997)) and intermixing methods.

Previous inventions (e.g. U.S. Pat. Nos. 8,114,918; 8,222,306; 8,283,030; 8,314,158; and 8,372,891) disclose that increasing the salt concentration within the bulk of the fully hydrated polyelectrolyte complex, by contacting it with a sufficiently high concentration of salt, renders the complex flowable without resorting to a change in temperature or other conditions. Under such flowable conditions the complex may be reshaped into a second persistent shape. Said shape persists in solutions of salt. Conversely, decreasing the salt concentration with the bulk of the polyelectrolyte complex is believed to cause the complex to revert to a non-flowable state. Advantageously, the transformation of the complex into a flowable material took place without recourse to elevated temperatures and without the requirement for organic solvents or acids or organic plasticizers. Accordingly, the dynamic mechanical properties of an article comprising the polyelectrolyte complex may be initially controlled by controlling the salt concentration during the preparation of the polyelectrolyte complex and then altered by increasing or decreasing the salt concentration of the solution contacting the article after preparation. Thus, for example, a flowable article may be prepared in the presence of high salt concentration, and then injected into a mold. Once the flowable article is in the mold, or has been removed from the mold, a concentration gradient may be applied by contacting the reshaped article with a solution having a lower salt concentration, which thereby causes salt located in the bulk of the article to diffuse out into the solution, making the article less flowable, thereby causing an increase in the modulus of the article, which is defined by the inner surfaces of the mold.

In general, the polyelectrolyte complex is formed by combining a predominantly negatively charged polyelectrolyte and a predominantly positively charged polyelectrolyte. In a preferred embodiment, the formation of the article starts with combining separate solutions, each containing one of the polyelectrolytes; in this embodiment, at least one solution comprises at least one predominantly positively-charged polyelectrolyte, and at least one solution comprises at least one predominantly negatively-charged polyelectrolyte. The formation of a polyelectrolyte complex, Pol⁺Pol⁻, by mixing individual solutions of the polyelectrolytes in their respective salt forms, Pol⁺A⁻ and Pol⁻M⁺, may be represented by the following equation:

Pol⁺A⁻+Pol⁻M⁺→Pol⁺Pol⁻+MA

where M⁺ is a salt cation, such as sodium, and A⁻ is a salt anion such as chloride. Pol⁻ and Pol⁺ represent repeat units on predominantly negatively charged and predominantly positively charged polyelectrolytes, respectively. According to the equation, the process of complexation releases salt ions into external solution, which are then part of the salt solution concentration.

The precipitates of polyelectrolyte complex, Pol⁺Pol⁻, formed by the reaction above are usually loose with much entrained water. The as-precipitated complex may be formed into the stored strain article or it may be allowed to densify or consolidate further by sitting for a period of time, or being mechanically worked. The material that is eventually used for the mechanical deformation step (to produce the stored strain article) is termed the “starting polyelectrolyte complex.”

Separate solutions containing the polyelectrolytes are preferably combined in a manner that allows the positively-charged polyelectrolyte(s) and the negatively-charged polyelectrolyte(s) to intermix. Intermixing the respective polyelectrolytes causes the in situ formation of a polyelectrolyte complex comprising an intermolecular blend of the positively-charged polyelectrolyte and the negatively-charged polyelectrolyte.

Individual polyelectrolyte solutions that are mixed may themselves comprise mixtures of polyelectrolytes of different chemical composition and/or molecular weight. For example, a solution may comprise two positive polyelectrolytes with two distinct chemical compositions. When the mixture of positive polyelectrolytes is mixed with the negative polyelectrolyte solutions the resulting complex will incorporate a blend of the two positive polyelectrolytes. Such a strategy is described for example in U.S. Pat. No. 7,722,752.

Inventions disclosed in U.S. Pat. Nos. 8,114,918; 8,222,306; 8,283,030; 8,314,158; and 8,372,891 describe and require that salt ions be present in the polyelectrolyte complex to render it sufficiently flowable for deformation and processing e.g. by extrusion through an orifice. Salt breaks the intermolecular ion pairing between polymers, allowing it to flow.

In theory, polyelectrolyte complexes that are not doped with salt are at their maximum crosslink density, i.e., at the maximum density of ion pair formation. It is known to the art that highly crosslinked polymers are not suitable for extrusion. For the present invention, it was discovered by accident that salt doping is not required to deform polyelectrolyte complexes as long as they remain fully hydrated during deformation.

It was further discovered that salt-free polyelectrolyte complexes when deformed while fully hydrated are significantly stronger (in the direction of the deformation) and tougher than salt-doped hydrated polyelectrolyte complexes.

The deformed polyelectrolyte complex article of the present invention may comprise a plurality of pores. The plurality or population of pores encapsulated in the polyelectrolyte complex article has at least one average transverse dimension, for example, a diameter, whose length ranges from about 100 nanometers to about 1000 micrometers, such as from about 0.5 micrometers (500 nanometers) to about 1000 micrometers, such as from about 1 micrometer to about 1000 micrometers, preferably from about 1 micrometer to about 100 micrometers, and more preferably from about 5 micrometers to about 100 micrometers. A transverse dimension comprises a distance from a point on one surface of the pore to another point on the opposing surface of the pore. When the pore is a sphere, the transverse dimension is identical to the diameter, this transverse dimension being sufficient to define the shape of the pore. When the pore is elongated, for example a prolate spheroid or an oblate spheroid, the transverse dimension may be either of the major or minor axes, these two transverse dimensions defining the shape of the pore.

The percentage pore volume, defined as the total volume of all the pores divided by the total volume of the article×100%. The total percentage volume of the porosity of the article is preferably from about 95 to about 1 percent. In some embodiments, pores comprised between about 10 and about 90% of the total volume of the article. For maximizing strength, the pore volume is preferably minimized, preferably to below 10% of the total volume of the article, more preferably below about 1% of the total volume of the article, even more preferably below about 0.1% of the total volume of the article. If pores are present, they may be elongated due to the strain applied to the article.

Polyelectrolytes for Complexes.

The charged polymers (i.e., polyelectrolytes) used to form the complexes are water and/or organic soluble and comprise one or more monomer repeat units that are positively or negatively charged. The polyelectrolytes used in the present invention may be copolymers that have a combination of charged and/or neutral monomers (e.g., positive and neutral; negative and neutral; positive and negative; or positive, negative, and neutral). Regardless of the exact combination of charged and neutral monomers, a polyelectrolyte of the present invention is predominantly positively charged or predominantly negatively charged and hereinafter is referred to as a “positively charged polyelectrolyte” or a “negatively charged polyelectrolyte,” respectively.

Alternatively, the polyelectrolytes can be described in terms of the average charge per repeat unit in a polymer chain. For example, a copolymer composed of 100 neutral and 300 positively charged repeat units has an average charge of 0.75 (3 out of 4 units, on average, are positively charged). As another example, a polymer that has 100 neutral, 100 negatively charged, and 300 positively charged repeat units would have an average charge of 0.4 (100 negatively charged units cancel 100 positively charged units leaving 200 positively charged units out of a total of 500 units). Thus, a positively-charged polyelectrolyte has an average charge per repeat unit between 0 and 1 and a negatively-charged polyelectrolyte has an average charge per repeat unit between 0 and −1. An example of a positively-charged copolymer is PDADMA-co-PAC (i.e., poly(diallyldimethylammonium chloride) and polyacrylamide copolymer) in which the PDADMA units have a charge of 1 and the PAC units are neutral so the average charge per repeat unit is less than 1.

Some polyelectrolytes comprise equal numbers of positive repeat units and negative repeat units distributed throughout the polymer in a random, alternating, or block sequence. These polyelectrolytes are termed “amphiphilic” polyelectrolytes. For examples, a polyelectrolyte molecule may comprise 100 randomly distributed styrene sulfonate repeat units (negative) and 100 diallyldimethylammonium chloride repeat units (positive), said molecule having a net charge of zero. If charges on one amphiphilic polymer associate with charges on another the material is considered a polyelectrolyte complex.

Some polyelectrolytes comprise a repeat unit that has both a negative and positive charge. Such repeat units are termed “zwitterionic” and the polyelectrolyte is termed a “zwitterionic polyelectrolyte.” Though zwitterionic repeat units contribute equal number of positive and negative repeat units, the zwitterionic group is still solvated and relatively hydrophilic. An example of a zwitterionic repeat unit is 3-[2-(acrylamido)-ethyldimethyl ammonio] propane sulfonate, AEDAPS. Zwitterionic groups are present on polyelectrolytes as blocks or randomly dispersed throughout the polymer chain. Preferably, polyelectrolytes comprise between about 1% and about 90% zwitterion units, and more preferably said polyelectrolyte comprises between about 10% and about 70% zwitterionic units. Preferred compositions of polyelectrolytes comprising zwitterionic repeat units also comprise between about 10% and about 90% non-zwitterionic charged repeat units.

The charges on a polyelectrolyte may be derived directly from the monomer units, or they may be introduced by chemical reactions on a precursor polymer. For example, PDADMA is made by polymerizing diallyldimethylammonium chloride, a positively charged water soluble vinyl monomer. PDADMA-co-PAC is made by the polymerization of a mixture of diallyldimethylammonium chloride and acrylamide (a neutral monomer which remains neutral in the polymer). Poly(styrenesulfonic acid) is often made by the sulfonation of neutral polystyrene. Poly(styrenesulfonic acid) can also be made by polymerizing the negatively charged styrene sulfonate monomer. The chemical modification of precursor polymers to produce charged polymers may be incomplete and typically result in an average charge per repeat unit that is less than 1. For example, if only about 80% of the styrene repeat units of polystyrene are sulfonated, the resulting poly(styrenesulfonic acid) has an average charge per repeat unit of about −0.8.

Examples of a negatively-charged synthetic polyelectrolyte include polyelectrolytes comprising a sulfonate group (—SO₃⁻), such as poly(styrenesulfonic acid) (PSS), poly(2-acrylamido-2-methyl-1-propane sulfonic acid) (PAMPS), sulfonated poly (ether ether ketone) (SPEEK), poly(ethylenesulfonic acid), poly(methacryloxyethylsulfonic acid), their salts, and copolymers thereof; polycarboxylates such as poly(acrylic acid) (PAA) and poly(methacrylic acid), polyphosphates, and polyphosphonates.

Examples of a positively-charged synthetic polyelectrolyte include polyelectrolytes comprising a quaternary ammonium group, such as poly(diallyldimethylammonium chloride) (PDADMA), poly(vinylbenzyltrimethylammonium) (PVBTA), ionenes, poly(acryloxyethyltrimethyl ammonium chloride), poly(methacryloxy(2-hydroxy)propyltrimethyl ammonium chloride), and copolymers thereof; polyelectrolytes comprising a pyridinium group such as poly(N-methylvinylpyridinium) (PMVP), including poly(N-methyl-2-vinylpyridinium) (PM2VP), other poly(N-alkylvinylpyridines), and copolymers thereof; protonated polyamines such as poly(allylaminehydrochloride) (PAH), polyvinylamine, polyethyleneimine (PEI); polysulfoniums, and polyphosphoniums.

Exemplary polyelectrolyte repeat units, both positively charged and negatively charged, are shown in Table I.

TABLE I
Polyelectrolyte Repeat Units

| Name | Structure |
|---|---|
| diallyldimethylammonium (PDADMA) | 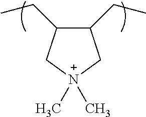 |
| styrenesulfonic acid (PSS) | 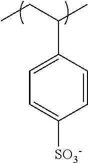 |
| N-methyl-2-vinyl pyridinium (PM2VP) | 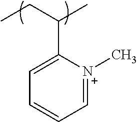 |
| N-methyl-4-vinylpyridinium (PM4VP) | 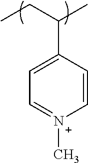 |
| N-octy1-4-vinylpyridinium (PNO4VP) | 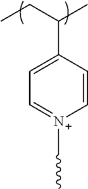 |
| N-methyl-2-vinyl pyridinium- co-ethyleneoxide (PM2VP-co-PEO) | 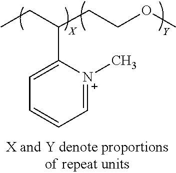 |
| acrylic acid (PAA) | 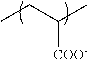 |
| allylamine (PAH) | 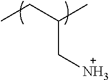 |
| ethyleneimine (PEI) | 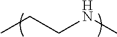 |

Further examples of polyelectrolytes include charged biomacromolecules, which are naturally occurring polyelectrolytes, or synthetically modified charged derivatives of naturally occurring biomacromolecules, such as modified celluloses, chitosan, or guar gum. A positively-charged biomacromolecule usually comprises a protonated sub-unit (e.g., protonated amines). Some negatively charged biomacromolecules comprise a deprotonated sub-unit (e.g., deprotonated carboxylates or phosphates). Examples of biomacromolecules which may be charged for use in accordance with the present invention include proteins, polypeptides, enzymes, DNA, RNA, glycosaminoglycans, alginic acid, chitosan, chitosan sulfate, cellulose sulfate, polysaccharides, dextran sulfate, carrageenin, glycosaminoglycans, sulfonated lignin, and carboxymethylcellulose.

Natural, or biological, polyelectrolytes typically exhibit greater complexity in their structure than synthetic polyelectrolytes. For example, proteins may comprise any combination of about 2 dozen amino acid building blocks, some charged, which are natural repeat units. Polymeric nucleic acids such as DNA and RNA may also comprise many different monomer repeat units (“nucleobases”). The sign and magnitude of the charge on proteins depends on the solution pH, as the charge on proteins is carried by weak acids, such as carboxylates (—COOH), or weak bases, such as primary, secondary, and tertiary amines. Thus, at high pH (basic conditions) amines are deprotonated and uncharged, and carboxylate groups are deprotonated and charged. At low pH (acidic conditions) amines are protonated and charged, and carboxylate groups are protonated and uncharged. For proteins, there is a pH at which there are equal numbers of positive and negative charges on the biomolecule, and it is thus electrically neutral. This is termed the isoelectric point, or pI. At pH above the isoelectric point, the protein has a net negative charge and at pH below pI, proteins bear a net positive charge. Proteins that tend to have a preponderance of positive charge at physiological pH, characterized by a high pI, are often termed “basic” proteins, and proteins with a low pI are called “acidic” proteins.

The molecular weight (number average) of synthetic polyelectrolyte molecules is typically about 1,000 to about 5,000,000 grams/mole, preferably about 10,000 to about 1,000,000 grams/mole. The molecular weight of naturally occurring polyelectrolyte molecules (i.e., biomacromolecules), however, can reach as high as 10,000,000 grams/mole. The polyelectrolyte solution typically comprises about 0.01% to about 50% by weight of a polyelectrolyte, and preferably about 1% to about 20% by weight.

Many of the foregoing polymers/polyelectrolytes, such as PDADMA and PEI, exhibit some degree of branching. Branching may occur at random or at regular locations along the backbone of the polymer. Branching may also occur from a central point and in such a case the polymer is referred to as a “star” polymer, if generally linear strands of polymer emanate from the central point. If, however, branching continues to propagate away from the central point, the polymer is referred to as a “dendritic” polymer. Branched polyelectrolytes, including star polymers, comb polymers, graft polymers, and dendritic polymers, are also suitable for purposes of this invention. Block polyelectrolytes, wherein a macromolecule comprises at least one block of charged repeat units, are also suitable. The number of blocks may be 2 to 5. Preferably, the number of blocks is 2 or 3. If the number of blocks is 3 the block arrangement is preferably ABA.

Many of the foregoing polyelectrolytes have very low toxicity. For example, poly(diallyldimethylammonium chloride), poly(2-acrylamido-2-methyl-1-propane sulfonic acid) and their copolymers are used in the personal care industry, e.g., in shampoos. Also, because some of the polyelectrolytes used in the method of the present invention are synthetic or synthetically modified natural polymers, their properties (e.g., charge density, viscosity, water solubility, and response to pH) may be tailored by adjusting their composition.

By definition, a polyelectrolyte solution comprises a solvent. An appropriate solvent is one in which the selected polyelectrolyte is soluble. Thus, the appropriate solvent is dependent upon whether the polyelectrolyte is considered to be hydrophobic or hydrophilic. A hydrophobic polymer displays less favorable interaction energy with water than a hydrophilic polymer. While a hydrophilic polymer is water soluble, a hydrophobic polymer may only be sparingly soluble in water, or, more likely, insoluble in water. Likewise, a hydrophobic polymer is more likely to be soluble in organic solvents than a hydrophilic polymer. In general, the higher the carbon to charge ratio of the polymer, the more hydrophobic it tends to be. For example, polyvinyl pyridine alkylated with a methyl group (PNMVP) is considered to be hydrophilic, whereas polyvinyl pyridine alkylated with an octyl group (PNOVP) is considered to be hydrophobic. Thus, water is preferably used as the solvent for hydrophilic polyelectrolytes and organic solvents such as ethanol, methanol, dimethylformamide, acetonitrile, carbon tetrachloride, and methylene chloride are preferably used for hydrophobic polyelectrolytes. Even if polyelectrolyte complexes are prepared by mixing organic-soluble and water-soluble polymers, the complex is preferably rinsed to remove organic solvents before it is reshaped according to the method described herein. Some organic solvents are hard to remove even with extensive rinsing. Therefore, the preferred solvent for polyelectrolyte complexation is water.

Examples of polyelectrolytes that are soluble in water include poly(styrenesulfonic acid), poly(2-acrylamido-2-methyl-1-propane sulfonic acid), sulfonated lignin, poly(ethylenesulfonic acid), poly(methacryloxyethylsulfonic acid), poly(acrylic acids), poly(methacrylic acids), their salts, and copolymers thereof; as well as poly(diallyldimethylammonium chloride), poly(vinylbenzyltrimethylammonium), ionenes, poly(acryloxyethyltrimethyl ammonium chloride), poly(methacryloxy(2-hydroxy)propyltrimethyl ammonium chloride), and copolymers thereof; and polyelectrolytes comprising a pyridinium group, such as, poly(N-methylvinylpyridium), and protonated polyamines, such as, poly(allylamine hydrochloride), polyvinylamine and poly(ethyleneimine).

Examples of polyelectrolytes that are soluble in non-aqueous solvents, such as ethanol, methanol, dimethylformamide, acetonitrile, carbon tetrachloride, and methylene chloride include poly(N-alkylvinylpyridines), and copolymers thereof in which the alkyl group is longer than about 4 carbon atoms. Other examples of polyelectrolytes soluble in organic solvents include poly(styrenesulfonates), poly(diallyldimethylammonium), poly(N-alkylvinylpyridinium), poly(alkylimidazoles), poly(vinylbenzylalkylammoniums) and poly(ethyleneimine) where the small inorganic counterion, such as, sodium, potassium, chloride or bromide, has been replaced by a hydrophobic counterion such as tetrabutyl ammonium, tetraethyl ammonium, tetraalkylammonium, alkylammonium, alkylphosphonium, alkylsulfonium, alkylimidazolium, alkylpiperidinium, alkylpyridinium, alkylpyrazolium, alkylpyrrolidinium, iodine, alkylsulfate, arylsulfonates, hexafluorophosphate, tetrafluoroborate, trifluoromethane sulfonate, hexyluorphosphate or bis(trifluoromethane)sulfonimide.

Preferred polyelectrolytes comprise rigid rod backbones, such as aromatic backbones, or partially aromatic backbones, including sulfonated polyparaphenylene, sulfonated polyetherether ketones (SPEEK), sulfonated polysulfones, sulfonated polyarylenes, sulfonated polyarylene sulfones, and polyarylenes comprising alkylammonium groups.

The charged polyelectrolyte may be a synthetic copolymer comprising pH sensitive repeat units, pH insensitive repeat units, or a combination of pH sensitive repeat units and pH insensitive repeat units. pH insensitive repeat units maintain the same charge over the working pH range of use. The rationale behind such a mixture of pH sensitive groups and pH insensitive groups on the same molecule is that the pH insensitive groups interact with other, oppositely-charged pH insensitive groups on other polymers, holding the polyelectrolyte complex together despite the state of ionization of the pH sensitive groups.

For example, poly(acrylic acids) and derivatives begin to take on a negative charge within the range of about pH 4 to about 6 and are negatively charged at higher pH levels. Below this transition pH range, however, poly(acrylic acids) are protonated (i.e., uncharged). Similarly, polyamines and derivative thereof take on a positive charge if the pH of the solution is below their pKₐ. As such, and in accordance with the present invention, the pH of a polyelectrolyte solution may be adjusted by the addition of an acid and/or base in order to attain, maintain, and/or adjust the electrical charge of a polyelectrolyte at the surface of, or within, a polyelectrolyte complex.

The state of ionization, or average charge per repeat unit, for polyelectrolytes bearing pH sensitive groups depends on the pH of the solution. For example, a polyelectrolyte comprising 100 pH insensitive positively charged units, such as DADMA, and 30 pH sensitive negatively charged units, such as acrylic acid, AA, will have a net charge of +100 at low pH (where the AA units are neutral) and an average of +100/130 charge per repeat unit; and a net charge of +70 at high pH (where 30 ionized AA units cancel out 30 of the positive charges) and an average of +70/130 charge per repeat unit. The different monomer units may be arranged randomly along the polymer chain (“random” copolymer) or they may exist as blocks (“block” copolymer). The average charge per repeat unit is also known as the “charge density.”

pH sensitive polyelectrolyte complexes comprise pH sensitive polymeric repeat units, selected for example, from moieties containing carboxylates, pyridines, imidazoles, piperidines, phosphonates, primary, secondary and tertiary amines, and combinations thereof. Therefore, preferred polyelectrolytes used in accordance with this invention include copolymers comprising carboxylic acids, such as poly(acrylic acids), poly(methacrylic acids), poly(carboxylic acids), and copolymers thereof. Additional preferred polyelectrolytes comprise protonatable nitrogens, such as poly(pyridines), poly(imidazoles), poly(piperidines), and poly(amines) bearing primary, secondary or tertiary amine groups, such as poly(vinylamines) and poly(allylamine).

To avoid disruption and possible decomposition of the polyelectrolyte complex, polyelectrolytes comprising pH sensitive repeat units additionally comprise pH insensitive charged functionality on the same molecule. In one embodiment, the pH insensitive repeat unit is a positively charged repeat unit selected from the group consisting of repeat units containing a quaternary nitrogen atom, a sulfonium (S⁺) atom, or a phosphonium atom. Thus, for example, the quaternary nitrogen may be part of a quaternary ammonium moiety (—N⁺RₐR_{b}R_{c} wherein Rₐ, R_{b}, and R_{c} are independently alkyl, aryl, or mixed alkyl and aryl), a pyridinium moiety, a bipyridinium moiety or an imidazolium moiety, the sulfonium atom may be part of a sulfonium moiety (—S⁺R_{d}Rₑ wherein R_{d} and Rₑ are independently alkyl, aryl, or mixed alkyl and aryl) and the phosphonium atom may be part of a phosphonium moiety (—P⁺R_{f}R_{g}Rₕ wherein R_{f}, R_{g}, and Rₕ are independently alkyl, aryl, or mixed alkyl and aryl). In another embodiment, the pH insensitive repeat unit is a negatively charged repeat unit selected from the group consisting of repeat units containing a sulfonate (—SO₃⁻), a phosphate (—OPO₃⁻), or a sulfate (—SO₄⁻).

Exemplary negatively charged pH insensitive charged repeat units include styrenesulfonic acid, 2-acrylamido-2-methyl-1-propane sulfonic acid, sulfonated lignin, ethylenesulfonic acid, methacryloxyethylsulfonic acid, sulfonated ether ether ketone, phosphate. Preferred pH insensitive negatively charged polyelectrolytes include polyelectrolytes comprising a sulfonate group (—SO₃⁻), such as poly(styrenesulfonic acid) (PSS), poly(2-acrylamido-2-methyl-1-propane sulfonic acid) (PAMPS), sulfonated poly (ether ether ketone) (SPEEK), sulfonated lignin, poly(ethylenesulfonic acid), poly(methacryloxyethylsulfonic acid), their salts, and copolymers thereof.

Exemplary positively charged pH insensitive repeat units include diallyldimethylammonium, vinylbenzyltrimethylammonium, vinylalkylammoniums, ionenes, acryloxyethyltrimethyl ammonium chloride, methacryloxy(2-hydroxy)propyltrimethyl ammonium, N-methylvinylpyridinium, other N-alkylvinyl pyridiniums, a N-aryl vinyl pyridinium, alkyl- or aryl imidazolium, sulfonium, or phosphonium. Preferred pH insensitive positively-charged polyelectrolytes comprising a quaternary ammonium group, such as poly(diallyldimethylammonium chloride) (PDADMA), poly(vinylbenzyltrimethylammonium) (PVBTA), poly(alkyammoniums), ionenes, poly(acryloxyethyltrimethyl ammonium chloride), poly(methacryloxy(2-hydroxy)propyltrimethyl ammonium chloride), and copolymers thereof; polyelectrolytes comprising a pyridinium group such as poly(N-methylvinylpyridinium) (PMVP), other poly(N-alkylvinylpyridines), and copolymers thereof.

For illustrative purposes, certain of the pH insensitive positively-charged moieties are illustrated below:

Pyridinium having the structure:

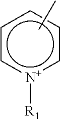

wherein R₁ is optionally substituted alkyl, aryl, alkaryl, alkoxy or heterocyclo. Preferably, R₁ is alkyl or aryl, and still more preferably R₁ is methyl;

Imidazolium having the structure:

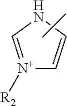

wherein R₂ is optionally substituted alkyl, aryl, alkaryl, alkoxy or heterocyclo. Preferably, R₂ is alkyl or aryl, and still more preferably R₂ is methyl;

Bipyridinium having the structure:

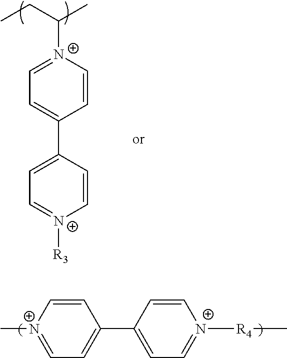

wherein R₃ and R₄ are optionally substituted alkyl, aryl, alkaryl, alkoxy or heterocyclo. Preferably, R₃ and R₄ are alkyl or aryl, and still more preferably R₃ is methyl.

The pH insensitive polyelectrolyte may comprise a repeat unit that contains protonatable functionality, wherein the functionality has a pKa outside the range of experimental use. For example, poly(allylamine) has protonatable amine functionality with pKa in the range 8-10, and is thus fully charged (protonated) if the experimental conditions do not surpass a pH of about 7.

Preferably, the pH insensitive groups constitute about 10 mol % to about 100 mol % of the repeat units of the polyelectrolyte, more preferably from about 20 mol % to about 80 mol %. Preferably, the pH sensitive groups constitute about 30 mol % to about 70 mol % of the repeat units of the polyelectrolyte.

Optionally, the polyelectrolytes comprise an uncharged repeat unit that is not pH sensitive in the operating pH range, for example, about pH 3 to about pH 9. Said uncharged repeat unit is preferably hydrophilic. Preferred uncharged hydrophilic repeat units are acrylamide, vinyl pyrrolidone, ethylene oxide, and vinyl caprolactam. The structures of these uncharged repeat units are shown in Table II. Preferred uncharged repeat units also include N-isopropylacrylamide and propylene oxide.

TABLE II
Neutral Repeat Units

| Name | Structure |
|---|---|
| Acrylamide | 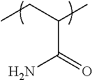 |
| Vinylpyrrolidone | 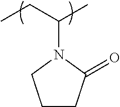 |
| Ethylene oxide | 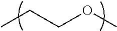 |
| Vinylcaprolactam | 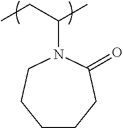 |

Protein adsorption is driven by the net influence of various interdependent interactions between and within surfaces and biopolymer. Possible protein-polyelectrolyte interactions can arise from 1) van der Waals forces 2) dipolar or hydrogen bonds 3) electrostatic forces 4) hydrophobic effects. Given the apparent range and strength of electrostatic forces, it is generally accepted that the surface charge plays a major role in adsorption. However, proteins are remarkably tenacious adsorbers, due to the other interaction mechanisms at their disposal. It is an object of this invention to show how surfaces may be selected to encourage or discourage the adsorption of proteins to strained polyelectrolyte complexes when they are used in vivo. Protein adsorption may be discouraged by incorporating polyelectrolytes comprising repeat units having hydrophilic groups and/or zwitterionic groups.

Polyelectrolyte complexes comprising zwitterions useful for preventing protein and/or cell adhesion have been described in U.S. Pat. Pub. No. 20050287111. It has been found that polymers comprising zwitterionic functional groups alone do not form polyelectrolyte complexes if they are employed under conditions that maintain their zwitterionic character. This is because the charges on zwitterionic groups do not exhibit intermolecular interactions. Therefore, preferred polymers comprising zwitterionic groups also comprise additional groups capable of intermolecular interactions, such as hydrogen bonding or ion pairing. More preferably, polyelectrolytes comprising zwitterionic groups also comprise charged groups that are not zwitterionic. Zwitterionic groups are present on polyelectrolytes as blocks or randomly dispersed throughout the polymer chain. Preferably, polyelectrolytes comprise between about 1% and about 90% zwitterions units, and more preferably said polyelectrolyte comprises between about 10% and about 70% zwitterionic units. Preferred compositions of polyelectrolytes comprising zwitterionic repeat units also comprise between about 10% and about 90% non-zwitterionic charged repeat units. Preferred zwitterionic repeat units are poly(3-[2-(acrylamido)-ethyldimethyl ammonio] propane sulfonate) (PAEDAPS) and poly(N-propane sulfonate-2-vinyl pyridine) (P2PSVP). The structures of these zwitterions are shown in Table III. Examples of other suitable zwitterionic groups are described in U.S. Pat. Pub. No. 20050287111, which is hereby incorporated by reference.

TABLE III
Zwitterionic Repeat Units

| Name | Structure |
|---|---|
| 3-[2-(acrylamido)- ethyldimethyl ammonio] propane sulfonate (AEDAPS) | 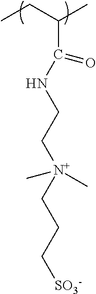 |
| N-propane sulfonate-2-vinyl pyridine (2PSVP) | 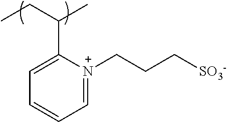 |

It has been disclosed (U.S. Pat. Pub. No. 20050287111) that films of polyelectrolyte complex prepared by the multilayering method are able to control the adsorption of protein. It is also generally known by those skilled in the art that hydrophilic units, such as ethylene oxide (or ethylene glycol), generally containing —C—C—O— repeat units, are effective in reducing the overall propensity of biological macromolecules, or biomacromolecules, to adsorb to surfaces (see Harris, Poly(ethylene glycol) Chemistry: Biotechnical and Biomedical Applications, Plenum Press, New York, 1992). Yang and Sundberg (U.S. Pat. No. 6,660,367) disclose materials comprising ethylene glycol units that are effective at resisting the adsorption of hydrophilic proteins in microfluidic devices. The ethylene oxide (or ethylene glycol) repeat units are preferably present as blocks within a block copolymer. Preferably, the block copolymer also comprises blocks of charged repeat units, allowing the material to be incorporated into a polyelectrolyte complex. Sufficient ethylene oxide repeat units are required to promote resistance to protein adsorption, but too many ethylene oxide units do not allow polyelectrolyte complexes to associate. Therefore, the preferred moles ratio of charged to neutral repeat units in a polyelectrolyte complex is from 10:1 to 1:4, and a more preferred ratio is 5:1 to 1:2.

Ethylene oxide (also termed oxoethylene) repeat units may also be employed in comb polymers, preferably with a main, charged chain comprising a plurality of at least one of the charged repeat units listed previously and oligomers or polymers of ethylene oxide units grafted to this main chain. Such an architecture is termed a comb polymer, where the charged backbone represents that backbone of the comb and the grafted ethylene oxide oligomers or polymers represent the teeth of the comb.

Preferably the location of the zwitterionic and/or polyethylene oxide repeat units is at the surface of the strained polyelectrolyte complex. In order to provide anti-biofouling properties to the strained polyelectrolyte complex the zwitterionic and/or polyethylene oxide repeat units are sorbed on the stressed polyelectrolyte complex article after extrusion, for example by exposing the article to a solution comprising a polyelectrolyte comprising zwitterionic or ethylene oxide repeat units. Alternatively, the zwitterionic or ethylene oxide functionality can be chemically grafted to the surface of the stressed polyelectrolyte complex using chemical grafting or coupling methods.

In some applications the surface of the strained polyelectrolyte complex is rendered bioadhesive, for example by the sorption of peptides (synthetic or natural) or proteins, such as fibronectin, comprising the RGD sequence of amino acids, as disclosed in U.S. Pat. Pub. No. 20030157260 and U.S. Pat. No. 6,743,521. In other embodiments the surface of the strained polyelectrolyte complex comprises 3,4-dihydroxyphenylalanine (DOPA) or catechol units, which are known to be bioadhesive. In further embodiments the surface of the stored strain polyelectrolyte complex further comprises reactive functional groups, such as aldehydes, ketones, carboxylic acid derivatives, anhydrides (e.g., cyclic anhydrides), alkyl halides, acyl azides, isocyanates, isothiocyanates, and succinimidyl esters. These groups react with amine groups found in biological tissue. Thus, an article comprising said groups adheres to tissue.

In one preferred embodiment, chemical crosslinking is introduced into the polyelectrolyte complex for stability after deformation. After deformation, for example by extrusion, an article may be treated with a difunctional crosslinking agent, such as XCH₂-φ-CH₂X, where X is a halogen (Cl, Br, or I) and φ is a phenyl group. The phenyl group may be replaced by another aromatic or aliphatic moiety, and easily-diplaceable groups, such as toluene sulfonate, may replace the halogen. A preferred crosslinking agent is a dihalogenated compound, such as an aromatic or aliphatic dibromide, which is able to alkylate residual unalkylated units on two adjoining polyelectrolyte chains.

Another preferred method of chemical crosslinking a polyelectrolyte complex after straining is heat treatment. For example, Dai et al. (Langmuir 17, 931 (2001)) disclose a method of forming amide crosslinks by heating a polyelectrolyte multilayer comprising amine and carboxylic acid groups. Yet another preferred method of introducing crosslinking, disclosed by Kozlovskaya et al. (Macromolecules, 36, 8590 (2003)) is by the addition of a carbodiimide, which activates chemical crosslinking. The level of chemical crosslinking is preferably between about 0.01% and about 50% as measured as a percentage of total ion pairs within the polyelectrolyte complex, and more preferably between about 0.1% and about 10% as measured as a percentage of total ion pairs within the polyelectrolyte complex.

Another method of chemical crosslinking of a strained polyelectrolyte complex is by photocrosslinking. Photocrosslinking may be achieved by the light-induced decomposition or transformation of functional groups, such as diarylbenzophenones, that form part of the polymer molecules. See, for example, Strehmel, Veronika, “Epoxies: Structures, Photoinduced Cross-linking, Network Properties, and Applications”; Handbook of Photochemistry and Photobiology (2003), 2, 1-110. See also Allen, Norman S., “Polymer photochemistry”, Photochemistry (2004), 35, 206-271; Timpe, Hans-Joachim “Polymer photochemistry and photocrosslinking” Desk Reference of Functional Polymers (1997), 273-291, and Smets, G., “Photocrosslinkable polymers”, Journal of Macromolecular Science, Chemistry (1984), A21 (13-14), 1695-1703. Alternatively, photocrosslinking of a polyelectrolyte complex may be accomplished by infusing the reformed polyelectrolyte complex with a small photoactive crosslinker molecule, such as diazidostilbene, then exposing the polyelectrolyte complex to light.

In some embodiments, the polyelectrolyte complex comprises further physical crosslinks created by hydrogen bonding. Hydrogen bonding is weaker than chemical bonding and occurs between a hydrogen bond donor and a hydrogen bond acceptor. Hydrogen bonds are minimally impacted by the presence of salt and thus the level of physical crosslinking due to hydrogen bonding remains substantially the same as the salt concentration is varied. Accordingly, the polyelectrolyte complex further comprises polymer repeat units capable of hydrogen bonding. Examples of hydrogen bond donor/acceptor pairs are presented in U.S. Pat. Nos. 6,740,409 and 7,470,449 as well as U.S. Pat. Pub. No. 20050163714.

Stress and Strain.

Stress is produced by mechanical force in one or two directions. In engineering terms, stress in a direction is defined as the force per unit cross section area of a material and has the same units as pressure (Pascals). Strain is the deformation of an object in response to the applied stress. It is usually given as the fractional change in dimension. For elastic materials, stress causes strain and vice versa. The Young's modulus of the elastic material is stress/strain. For elastic materials, when stress is removed (i.e. goes to zero), so is strain (goes to zero).

Some materials, when strained under an external stress, do not recover their initial dimensions when the stress is removed, even after waiting. This is non-elastic flow behavior. Typically, irreversible viscous flow has occurred, as when polyelectrolyte complexes are reshaped by a mechanical force in the presence of salt. In another example, a ball of pasta dough forced through an extruder creates spaghetti. The spaghetti cannot be made to shorten or revert to a ball of dough, even if overcooked in boiling water.

Other materials store strain when they are deformed by a mechanical force. For example, the material is strained by an applied stress. When the stress is removed, the material does not recover its original dimensions. However, on an external stimulus, some of the original dimension is recovered. In other words, the strain is stored and then released later on by a stimulus. A good example of stored-strain material is heat-shrink tubing. Heat shrink tubing, widely used in electronics, is a sleeve of plastic which has been processed to store strain at room temperature. When the tubing is heated the strain is released and the tubing shrinks around the wiring is has been placed over. In this case the stimulus for releasing the stored strain is temperature.

It is known that if materials melt or soften surface tension is enough to change the shape of the material. For example, a piece of rough wax makes a smooth object when it melts. This is not considered stored strain for the purposes of the present invention. In another example, Dubas et al. (Langmuir, 17, 7715 (2001)) describe how the surface of rough polyelectrolyte multilayers, which are ultrathin films (less than 1 μm thick) of polyelectrolyte complexes, may be smoothed by exposure to high salt concentration. Again, this is not an example of stored strain. The morphology change is driven by surface tension and the multilayer is too small for any useful work to come from the shape change. Accordingly, the stored strain polyelectrolyte complex articles are preferably thicker than multilayers, i.e., preferably more than 1 μm thick, more preferably more than 10 μm thick. In order to have substantial mechanical properties the stored strain article is preferably greater than 50 micrometers thick: for example, a sheet of polyelectrolyte complex more than 50 micrometers thick or a fiber of complex more than 50 micrometers in diameter or a tube of polyelectrolyte complex wherein the wall of the tube is more than 50 micrometers thick. For practical purposes the article is preferably not more than 1 meter in a second dimension, although in a third dimension the length could be much longer, especially if the article is formed by extrusion. For example, a ribbon of strained polyelectrolyte complex could be 100 micrometers thick and 20 cm wide and 100 meters long. A tube could have a wall thickness of 1 mm, a total diameter of 10 mm and a length only limited by the amount of material for an extrusion run. Similarly, a fiber of complex could be 0.5 mm in diameter and several meters long, as illustrated in the example where the fiber is extruded and taken up on a takeup reel.

For strain to be stored in an article comprising polyelectrolyte complex the article must first be strained by applying a force. Said force deforms the article, i.e., changes the shape of the article. Various methods to apply mechanical force to deform a sample of polymer are known to the art and include extrusion, compression, extension, bending, twisting, wrapping, spiraling, expanding, and stretching polyelectrolyte complex in one or two dimensions.

A material in a stored strain state is not allowed to flow. If it did flow the strain would slowly be released. Materials in a state showing significant viscous stress-relaxation are not suitable for creating stored strain. Thus, there is an apparent contradiction: materials must be able to flow to be formed into an article (e.g. by extrusion) but the same flow property is not good for storing strain. Therefore, it cannot be known a priori whether a flowable material might be a good stored-strain material.

It is believed that straining a polyelectrolyte complex article leads to molecular orientation along the strain direction. Molecular orientation is characterized by a Herman's orientation function, f, where f=0 corresponds to nonoriented (random direction) chains and f=1 corresponds to fully oriented (fully aligned in the strain direction) chains. In many polymers, orientation causes an increase in modulus along the strain direction. In many polymers, orientation leads to anisotropic physical properties. For example, polarizing plastic lenses may be produced by straining some polymers.

It can be difficult to measure molecular orientation. Therefore, an operational definition of stored strain is used here. Stored strain is defined as the ratio of an article dimension in the strained dimension, before and after the stored strain is completely released. For example, a rod comprising stored strain polyelectrolyte complex is produced by extrusion. The length of the rod is x cm. The strain is fully released by any of the stimulii described herein and the rod shrinks to a length of y cm (meanwhile, the rod becomes thicker). The stored strain is x/y. In another example, a tube of stored strain polyelectrolyte complex of length m cm is produced by stretching a tube comprising polyelectrolyte complex along the tube length, then drying the tube. When the strain is released by any of the methods described herein the tube shrinks to length n cm. The stored strain is m/n. In another example a tube comprising polyelectrolyte complex is strained in the radial direction to a radius of p cm. When the strain is released by any of the stimuli described herein the radius shrinks to q cm. The stored strain is p/q. The ratios x/y, m/n, p/q may be termed the stored strain factor or the stored strain ratio.

For sufficient enhancement of materials properties, such as Young's modulus and toughness, and for sufficient response to a stimulus in order to release the stored strain, the preferred stored strain factor is greater than 2, more preferably greater than 3. For improving mechanical properties such as toughness, the maximum stored strain which may be achieved is preferred. In the example below stored strain of up to 5 is described. If possible, even higher stored strains are preferred. The maximum stored strain is dictated by the materials properties, the geometry of the extrusion and the mechanical performance of the extruder. For example, if the extruder could extrude faster (more grams of material per second) with a higher pressure it is likely that the polymer molecules would be better aligned and the stored strain would be higher. At the same time, the take-up reel could be rotated faster which helps elongate the fiber. The polyelectrolyte complex will retain the shape imparted by the applied stress, in the absence of a stimulus sufficient to release the strain, for a duration of at least about 10 minutes, preferably at least about 60 minutes, preferably at least about 7 days, and more preferably, indefinitely.

It has been discovered that stored strain polyelectrolyte complexes have improved mechanical properties in comparison to the same material that does not have stored strain. This is believed to be a result of enhanced molecular orientation of the polymer chains.

A stored strain polyelectrolyte complex of the present invention may have a “toughness” of at least about 1 MJ·m⁻³, preferably at least about 3 MJ·m⁻³.

Yet another improved property mentioned in the examples is the Young's modulus. Accordingly, a stored strain polyelectrolyte complex of the present invention may have a Young's modulus of at least about 500 MPa, preferably at least about 2000 MPa.

Additionally, a fiber made from stored strain polyelectrolyte complex of the present invention may be capable of being bent at least about 90° from its starting orientation, such as at least about 180°.

In one aspect of this invention the molecular orientation created as a result of straining is preserved by chemically crosslinking the polyelectrolyte complex during or after applying a mechanical force. Chemical crosslinking, which forms covalent bonds between polymer molecules, counteracts the effects of the stimulus or other mechanisms which lead to gradual release of the stored strain, and therefore the gradual loss of molecular orientation and therefore to gradual loss of strength (Young's modulus).

Chemical crosslinking that occurs as the stored strain article is being produced by extrusion is an example of reactive extrusion. Reactive extrusion is described in “Reactive Extrusion: Principles and Practice” by M. Xanthos (Oxford Univ. Press, 1992). A preferable reactive group is the anhydride. A preferred reactive extrusion during the forming of a strained polyelectrolyte complex article uses at least one polyelectrolyte comprising at least one of the charged repeat units described recently and an anhydride, and at least one polyelectrolyte comprising a repeat unit that reacts with an anhydride. For example, the first polyelectrolyte comprises styrene sulfonate repeat units and (random or alternating) maleic anhydride repeat units and a second polyelectrolyte comprises amine repeat units. During the reactive extrusion the anhydride reacts with the amine group. Anhydrides tend to be deactivated by water, and thus cannot be stored wet. As an alternative example, reactive extrusion may be performed on a starting polyelectrolyte complex of polyelectrolyte comprising repeat units comprising carboxylate functionality (such as a polyacrylic acid) and polyelectrolyte comprising repeat units comprising amine functionality (such as polyvinylamine or polyallylamine). Heat treating a complex of polycarboxylic acids and polyamines yields amide crosslinks. Other reactive functionalities are suitable for introducing crosslinks, for example wherein the first polyelectrolyte comprises alkenes and the second comprises thiols.

Hydration.

The starting polyelectrolyte complex is preferably fully hydrated when a mechanical force is applied to it to produce the stored strain polyelectrolyte complex. Since polyelectrolyte complexes may comprise pores it is not simply the total water content but the water hydrating the polyelectrolyte ion pairs that is important. Full hydration is achieved when the polyelectrolyte complex is contacted by water and equilibrium water uptake is allowed. The full hydration level is the equilibrium amount of water hydrating the polyelectrolyte ion pairs. Equilibrium uptake can be slow, therefore is it preferable that the starting polyelectrolyte complex not be allowed to dry after precipitation.

It becomes much more difficult to process, e.g., by extrusion, a starting polyelectrolyte complex if it has less than the full, or maximum or equilibrium water content. Therefore, a starting polyelectrolyte complex that is allowed to dry even slightly may prove to be unprocessible.

In order to maintain the starting polyelectrolyte complex in a hydrated state as it is reformed by a mechanical force into the stored strain polyelectrolyte complex article it is preferred to establish a wetting film of water on the starting polyelectrolyte before processing. A small excess of free liquid water (i.e., water not hydrating the polyelectrolyte molecules) is advantageous. Therefore, it is advantageous that the starting polyelectrolyte complex has the consistency of cottage cheese where pockets of water can be trapped. These trapped pockets or pores of water ensure full hydration of complex as it is deformed to the stored strain state. In terms of weight percent, it is preferable to have at least 10 weight percent more water than required for full hydration level in the starting complex more preferably, between 10 and 200 weight percent more water than required for full hydration level in the starting complex. Too high a water level may produce defects during extrusion e.g. by creating water vapor and steam.

Doping Level.

As stated above, doping of the polyelectrolyte complex affects the elastic and dynamic mechanical properties of the article comprising the complex, such as, for example, the elastic and complex shear modulus. It has been observed that doping by increasing the salt concentration decreases the article's shear modulus, G. Conversely, decreasing the salt concentration increases G, making the article stiffer.

The process of doping is defined as the breaking of polymer/polymer ion pair crosslinks by salt ions entering the polyelectrolyte complex. Salt ions electrically compensate the charges on the polyelectrolytes. In such compensation, the salt ions are termed counterions and are paired with polyelectrolyte repeat units of opposite charge. Salt ions residing in pores or paired with other salt ions or present as crystals are not considered to be doping the polyelectrolyte complex and do not contribute to the doping level. The level or density of doping is therefore inversely related to the crosslink density. The breaking of ion pair crosslinks by doping is reversible and under thermodynamic control. In contrast, chemical crosslinks are usually irreversible.

The doping level of polyelectrolyte complexes is created and maintained by contacting the complex with a solution comprising salt ions of a specific concentration. Equilibration of the polyelectrolyte complex in the salt solution in which the complex is immersed may be fairly rapid, with durations typically on the order of between about 10 minutes and about 60 minutes per millimeter thickness of the polyelectrolyte complex article.

The extent to which ion pair crosslinks have been replaced by salt counterions within the bulk of the article comprising polyelectrolyte complex may be quantified in terms of a doping level or doping level ratio, determined by dividing the sum of the ionic charge provided by salt ions acting as polyelectrolyte counterions by the sum of charge provided by the polymer repeat units. This ratio may be expressed in terms of a doping level percentage by multiplying the doping level ratio by 100. The lowest doping level is 0.0 (0%) wherein all the positively charged polyelectrolyte repeat units are paired with all the negatively charged polyelectrolyte repeat units, which corresponds to the maximum level (100%) of ionic crosslinking. The highest doping level is 1.0 (100%), where all charged polyelectrolyte repeat units are paired with a salt ion. When the doping level is 1.0 the polyelectrolytes are dissociated: phase separation can occur between components; additives can phase separate, and solutions do not maintain their shape when reformed. At a doping level of 1.0, the polyelectrolyte complex is dissolved, or maintained in solution, as described in U.S. Pat. No. 3,546,142.

The doping level can be measured, for example by infrared absorption spectroscopy (see e.g., Farhat and Schlenoff, Langmuir 2001, 17, 1184; and Farhat and Schlenoff, Journal of the American Chemical Society, 2003, vol. 125, p. 4627.)

To illustrate a doping level calculation, suppose that a simple polyelectrolyte complex comprises a blend of one positively charged polyelectrolyte having 100 positively charged repeat units paired with one negatively charged polyelectrolyte having 100 negatively charged repeat units. Such a polyelectrolyte complex therefore has a total charge provided by the charged repeat units of 200. The maximum number of ionic crosslinks is 100. This polyelectrolyte complex may be doped with salt ions which become associated with the charged repeat units. For example, if 10 sodium ions are associated with 10 negatively charged repeat units and 10 chloride ions are associated with 10 positively charged repeat units, the sum of charges provided by the salt ions is 20, and 10 ionic crosslinks have been broken. The doping level is a ratio calculated by dividing the sum of charges of the salt ions paired with polyelectrolytes by the sum of charges from the repeat units, i.e., 20/200=0.1, or 10%, stated as a doping level percentage. By way of further example, if 5 calcium ions (charge 2+) are associated with 10 negatively charged repeat units and 10 chloride ions are associated with 10 positively charged repeat units, the sum of charges provided by the salt ions is 20 (=5×2 for the calcium+10 for the chloride) and the doping level ratio is 20/200=0.1, or 10%, stated as a doping level percentage. To achieve these doping levels, the article comprising the polyelectrolyte is preferably maintained in contact with a solution of the doping salt in water. The salt concentration employed during preparation and compaction includes those ions liberated from the polyelectrolytes by complexation.

It has been shown quantitatively that the mechanical properties of articles comprising polyelectrolyte complex are influenced by the doping level. For example, Jaber and Schlenoff (e.g., see Journal of the American Chemical Society, 2006, vol. 128, p. 2940 and also U.S. Pat. No. 8,206,816) analyzed the mechanical properties of articles comprising nonporous polyelectrolyte complexes using classical theories of rubber elasticity. The elastic modulus of articles comprising nonporous polyelectrolyte complexes decreased as they were doped with salt ions. In the doping level range studied, which was about 0 to about 0.4, the articles were elastically deformed, meaning that they regained their original shape when the deforming force was removed.

It is an object of the present invention to form a stored strain polyelectrolyte complex which keeps its shape before a stimulus is applied. Hence, articles shaped or reshaped by applying a force to deform the article must not relax back to their original shape before the stimulus is applied. For example, a hydrated polyelectrolyte complex article may be deformed by a force. When the force is removed the article may regain its original form, such as when a polyelectrolyte complex is strained within the range of elastic behavior. In this range the polyelectrolyte complex behaves as a damped elastic material and exhibits a viscoelastic response to stress or strain.

The preferred doping level for shaping a polyelectrolyte complex article disclosed in U.S. Pat. No. 8,283,039 was claimed to be critically important. In order to shape a polyelectrolyte complex article into a persistent shape the doping level was required to be sufficiently high. In U.S. Pat. No. 8,222,306, describing compaction of starting polyelectrolyte complex by ultracentrifugation, a preferred solution salt concentration of 1.0M was provided and the preferred salt was NaCl. U.S. Pat. No. 8,283,030 disclosed a doping level of at least 0.5 for forming a polyelectrolyte complex article into a persistent shape. Preferred doping levels were given as between 0.6 and 0.990 and more preferably between 0.7 and 0.990. Stated in terms of a percentage, the doping level was preferred to be between about 60% and about 99.00, more preferably between about 70% and about 99.00.

In accordance with the present invention, the preferred level of doping of the starting polyelectrolyte complex is close to zero. That is, doping is not preferred and the starting complex is then termed “undoped.” Without being held to a particular theory, it is believed that a lack of salt doping preserves the maximum stored stress.

Undoped polyelectrolyte complexes are obtained by soaking polyelectrolyte complexes comprising stoichiometric amounts of positive and negative polyelectrolyte repeat units in water. It is not possible to remove all ions, as some ions remain trapped. The concentration of trapped ions is lower for polyelectrolytes which are mixed better. However, for practical purposes, the existence of trace amounts of ions does not affect the preferred properties of the final stored strain polyelectrolyte. In some polyelectrolyte complexes, counterions cannot be detected. Of course, whether trace ions are seen or not depends on the experimental methods used to measure them. The maximum allowable doping level is 0.1, or 10%. Preferably, the doping level is less than 0.01, or less than 1%, which is considered trace for the purposes of this invention.

Methods of Forming.

The polyelectrolyte is preferably maintained in a fully hydrated state during the method of forming the present invention preferably by contact with water. In the fully hydrated state chunks, pellets, pieces or other shapes or articles of starting complex are fully swollen with water, that is their water content approaches the maximum it would achieve when immersed in water under the conditions of forming. Pieces of starting complex that are fully hydrated, undoped and wetted by a film of water are suitable for the present invention. Because dried pieces of polyelectrolyte complex are difficult to rehydrate, it is preferred that the compact polyelectrolyte complex materials be prepared by coprecipitation of individual polyelectrolytes and maintained in a hydrated state, preferably in contact with water.

In one preferred embodiment, the shape of the article at the end of the reforming step is defined by the contours of a mold, in the case where the doped polyelectrolyte complex is forced into a mold.

In another preferred embodiment, the doped polyelectrolyte is extruded through an orifice, which defines the shape of the cross section of the reshaped article, such as rod, fiber, tape or tube. Methods known to the art for extruding materials, such as forcing materials through a die or orifice via a piston or a screw, are suitable. The orifice may be of any geometry known to the art, including those geometries that enhance the alignment of high-aspect-ratio fillers during the extrusion step. The orifice and other components are preferably made from corrosion-resistant materials, such as stainless steel, plastic or ceramic. For a screw extruder, a continuous form may be produced as long as pieces of polyelectrolyte complex are fed into the extruder continuously.

In yet another preferred embodiment, a pattern is embossed into an article of stored strain polyelectrolyte complex at a preferred low doping level. Embossing is performed with a metallic, polymeric, or ceramic material with features from the nanometer to the millimeter size range. Such a pattern may be quite intricate, the reformed polyelectrolyte complex article faithfully reproducing the features of the embossing pattern. For example, a microchannel or a series of microchannels may be embossed into the polyelectrolyte complex. In another example, a series of features representing bits of data for storage may be embossed into the polyelectrolyte complex. For embossing purposes, the stored strain polyelectrolyte complex is preferably planar.

The temperature is advantageously increased during the processing of starting polyelectrolyte complexes into stored strain complexes.

General Additives.

Solid additives that may be incorporated into the polyelectrolyte complex are typically known to the art to modify the physical properties of materials. Additives include fillers and/or reinforcing agents and/or toughening agents, such as inorganic materials such as metal or semimetal oxide particles (e.g., silicon dioxide, aluminum oxide, titanium dioxide, iron oxide, zirconium oxide, and vanadium oxide), clay minerals (e.g., hectorite, kaolin, laponite, attapulgite, montmorillonite), hydroxyapatite or calcium carbonate. For example, nanoparticles of zirconium oxide added to a polyelectrolyte solution or complex solution tend to improve the abrasion resistance of the article. See Rosidian et al., Ionic Self-assembly of Ultra Hard ZrO₂/polymernanocomposite Films, Adv. Mater. 10, 1087-1091 and U.S. Pat. No. 6,316,084. If the stored strain polyelectrolyte complex article comprises magnetic particles having at least one dimension in the size range between 2 nanometers and 100 micrometers the article may be manipulated with a magnetic field. High aspect ratio fillers are preferred for stiffening or strengthening an article at a relatively low fill loading. Preferred high aspect ratio additives include, metal fibers, inorganic platelets such as calcium carbonate or calcium phosphate (such as hydroxyapatite), needle-like clay minerals, such as attapulgite and halloysite, and carbon-based fibers such as carbon fibers or single or multiwalled carbon nanotubes or graphene. Other high aspect ratio materials having at least one dimension in the 1 nanometer to 100 micrometer range are suitable additives. Such high aspect ratio materials include polymer fibers, such as nylon, aramid, polyolefin, polyester, cotton, and cellulose fibers, as well as cellulose nanofibers. Biodegradable fibers are preferred when the stored strain polyelectrolyte complex article comprises biodegradable polyelectrolytes. The weight % of additives in the polyelectrolyte complex article depends on many factors, such as the aspect ratio and the degree of modification of physical properties required. Accordingly, the solid additives may comprise between about 1 wt % and 90 wt % of the polyelectrolyte complex article.

Preferably, additives are added prior to the preparation of the starting polyelectrolyte complex feed material. Negatively charge additives are preferably combined with solutions comprising negative charged polyelectrolytes prior to mixing with solutions comprising positively charged polyelectrolytes so that the additives and polyelectrolytes do not associate prematurely. Additives and individual polyelectrolytes are preferably thoroughly mixed in solution first under shear flow (as created by stirring or a homogenizer) with the proviso that the shear rate should not be sufficient to break up the polymer chains. If however, the polyelectrolyte stabilizes and assists in the dispersion of the additive it may be preferable to first mix additive and polyelectrolytes of opposite charge. For example, nanotubes can sometimes be dispersed better in solution if they are “wrapped” with polymers.

For physiological applications of the stored strain polyelectrolyte complex article, bioactive additives such as pharmaceuticals may be added during, or after the method of the present invention. For example, articles that are to be implanted in vivo may optionally further comprise antibacterial agents and/or anti-viral agents and/or anti-inflammation agents and/or antirejection agents and/or growth hormones and/or growth factors. These additives respectively aid in reducing infection, inflammation or rejection of the implanted article and encouraging tissue proliferation. Examples of antibiotics are well known to the art and are to be found in E. M. Scholar, The Antimicrobial Drugs, New York, Oxford University Press, 2000 or the Gilbert et al., The Stanford Guide to Antimicrobial Therapy, Hyde Park, Vt., 2000, or the R. Reese, Handbook of Antibiotics, Philadelphia, Lippincot, 2000. Antibacterial agents include silver including silver nanoparticles. Other additives are known to the art for promoting various biomedical properties. These include paclitaxel, seratonin, heparin, and anticlotting factors. Unlike additives used to modify the physical properties of the polyelectrolyte complex article, additives with biological or biomedical activity are typically added in lower concentration. Accordingly, such additives preferably comprise between 0.0001% (1 μg/g) and 5% by weight of the polyelectrolyte complex article. The concentration of the additive is typically adjusted to obtain the optimum physiological response.

Additives providing structural properties are preferably mixed with one of the constituent polyelectrolyte solutions that are used to prepare the polyelectrolyte complex. The advantage of introducing additives prior to precipitation is that the additives are incorporated more uniformly throughout the polyelectrolyte complex. Additives providing biological or bioactive properties are either mixed with one of the constituent polyelectrolyte solutions before the stored stress complex is prepared or they are sorbed into the surface of the complex after the stored stress complex is formed. If biologically active additives lose their activity on exposure to the temperature used while forming the stored strain polyelectrolyte complex the additive is preferably sorbed into the complex after it is formed (e.g. by extrusion).

Biocompatibility.

It has been shown that certain polyelectrolytes or polymers are biocompatible. For example, a biocompatible polyelectrolyte multilayer, on which smooth muscle cells were grown, has been described in U.S. Pat. Pub. No. 2005/0287111, which is herein incorporated by reference. This multilayer comprised fluorinated polyelectrolyte complex, on which cells grow. However, the cells do not consume the fluorinated material. In one aspect of the present invention, therefore, the stored strain polyelectrolyte complex article further comprises a surface stratum of fluorinated polyelectrolyte. The surface stratum is preferably obtained by immersing the stored strain polyelectrolyte complex article in a solution of fluorinated polyelectrolyte. The process may be repeated with alternating positive and negative fluorinated polyelectrolytes to obtain a thicker surface stratum.

Bioinertness.

It has been shown that a polyelectrolyte complex film comprising a zwitterion repeat unit has bioinert properties, i.e., the adsorption of proteins, cells and other biological materials is minimized on the film. (Examples are provided in U.S. Pub. No. 2005/0287111). Therefore, in one aspect of the present invention, the stored strain polyelectrolyte complex article further comprises a surface stratum comprising polyelectrolytes comprising zwitterionic repeat units. Other bioinert materials are known to the art, such as poly(ethylene glycols), PEG. Therefore, in one aspect of this invention, the polyelectrolyte complex article further comprises a surface stratum of PEG.

Other biological materials are known to be biocompatible, such as serum albumin. In one embodiment, the stored strain polyelectrolyte complex article may be coated with serum albumin on exposure to in vivo conditions (i.e. following implant).

Biodegradation.

In another aspect of the present invention, the stored strain polyelectrolyte complex comprises units known to degrade in a biological environment. Said degradation may be a result of ambient chemical hydrolysis of parts of the polyelectrolyte chain, such as an ester group, or degradation may be the result of enzymatic activity, such as promoted by bacteria. Examples of hydrolyzable groups on polyelectrolyte chains are provided in U.S. Pat. Pub. No. 20120065616 and references therein. Natural polyelectrolytes, such as glycosaminoglycans, or synthetically modified natural polyelectrolytes, such as chitosan, or synthetic polyelectrolytes comprising natural repeat units, such as polyglutamic acid and polylysine, are suitable for making biodegradable stored strain polyelectrolytes.

Stimuli for Strain Release.

Release of stored strain in a polyelectrolyte complex article as the result of a stimulus is characterized by a decrease in at least one dimension. For example, the stored strain in a rod of strained polyelectrolyte complex may be released by immersion in an aqueous solution of salt (the stimulus), whereupon the rod shortens.

Preferred stimuli include, individually or in combination, heating, solvation (e.g. hydration), a pH change, and doping by a solution comprising salt. For example, as described below, a polyelectrolyte complex comprising no salt, strained by extrusion, releases its stored strain on exposure to a solution comprising salt or exposure to hot water.

In preferred applications of stored strain polyelectrolyte complexes, stored stress not simply released by room temperature hydration. As seen in the examples below, stored stress articles comprising polyelectrolyte complex relax only slightly when fully hydrated by immersion in water at room temperature. Salt and/or heat is needed to provide the appropriate stimulus for releasing the stored strain.

Preferred salts for use as stimuli to release stored strain include all those salts capable of doping the polyelectrolyte complex. Yet more preferred is NaCl. The concentration of salt is preferably selected to promote the rate and extent of stored strain release. In physiological conditions, the preferred salt concentration is about 0.15 M NaCl, preferably at a pH of about 7.

When using solvation as a stimulus to release stored strain the preferred solvent is water. The stored strain article is preferably immersed in the solvent to stimulate strain release.

Heat as a stimulus is preferably used in combination with solvation, preferably full hydration, and optionally in combination with doping by salt. The preferred temperature range for heat as a stimulus is 20° C. to 100° C. when the heating is done in conjunction with solvation. Heat is provided via any method known, including heat lamps, or locally heating strained polyelectrolyte complex loaded with nanoparticles, such as iron oxide, capable of absorbing radiofrequency radiation to increase sample temperature. Optionally, heat energy can be provided to a strained polyelectrolyte complex comprising a molecule, such as a dye, or a particle, such as gold nanoparticles, capable of absorbing electromagnetic radiation. For physiological applications, said dye or nanoparticle preferably absorbs light in the red-near IR range (600-1200 nm).

Exposure of a stored strain polyelectrolyte complex comprising redox units to reducing or oxidizing conditions also serves as a stimulus for releasing stored strain. For example, if one or more of the polyelectrolytes comprising the stored strain complex comprises redox units and the redox state is changed by chemical or electrochemical methods the strain may be released. Examples of redox units include bipyridiniums and metal-organic centers such as ferrocene, ruthenium complexes, and osmium complexes. Other examples of redox units include conducting polymer units such as pyrrole, aniline, and thiophene. Yet other examples of redox units include disulfides, which are reduced to thiols.

### EXAMPLES

The following non-limiting examples are provided to further illustrate the present invention.

Materials and Methods.

Poly(4-styrenesulfonic acid) was from AkzoNobel (VERSA TL 130, MW of 200,000 g/mol), and poly(diallyldimethylammonium chloride) from Ondeo-Nalco (SD 46104, with MW of 410,000 g/mol). Sodium chloride (Aldrich) was used to adjust solution ionic strength. Deionized water (Barnstead, E-pure, Milli-Q) was used to prepare all solutions.

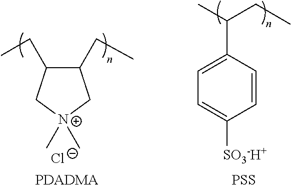

Solutions of PSS and PDADMA were prepared at a concentration of 0.125M with respect to their monomer units, neutralized to pH 7 with NaOH and their ionic strength adjusted (usually to 0.25M NaCl). Typically, 1 L of each was poured simultaneously into a 3 L beaker. 1 L of 0.25 M NaCl, used to rinse the flasks, was added to the precipitate. The mixture was stirred with a magnetic stirrer for about 30 min and the precipitated PEC was decanted and washed with 1 L of 1M NaCl. The PEC was chopped into pieces between 5 mm and 10 mm large then soaked in 1.0 M NaCl for 24 hr. The salt solution was strained off and excess liquid removed from the PEC pieces by rapid dabbing with a paper towel. The PEC was introduced, still fully hydrated, into the hopper of a Model LE-075 laboratory extruder from Custom Scientific Instruments, Inc. The following extruder parameters were selected by trial and error: rotor temperature; header temperature; gap space; rotor speed; and polyelectrolyte complex feed rate. The extruded complex was continuously collected on a Model CSI-194T takeup reel with a 3 cm diameter drum. These parameters allowed the extrusion of fiber at approximately 2 g min-1.

To determine the salt content of the polyelectrolyte complexes, thermogravimetric analysis (TGA) was performed with a SDT Q600 TGA from TA Instruments. Prior to thermal analysis, samples were dried for 24 h at 90° C. in vac and gently ground.

Mechanical properties of extruded polyelectrolyte complexes was measured via stress relaxation using a TH2730 (Thümler GmbH) tensile testing unit equipped with a 100N load cell. To remove residual stress induced by extrusion, samples were first immersed in 1M NaCl solution for 24 h. Samples were then soaked in solutions of various [NaCl] for 24 h prior to mechanical testing. Samples of diameter 1 mm and length 20 mm were stretched to a strain, ε, of 2% at a speed of 10 mm min-1 and the relaxation in stress recorded. Stress, σ, which relaxed to an “equilibrium” value, σₒ, was recorded vs. time. The equilibrium modulus, E₀, is given by E₀=Gσ₀/ε

Strain to break measurements were carried out on annealed samples at a stretching speed of 10 mm min⁻¹. Samples were cut into dogbone shapes of dimension 20 mm×15 mm. The toughness was calculated by integrating the area under the stress-strain (to failure) curve (ε_{f}′):

$$Toughness = {\int }_{0}^{{ɛ}_{f}^{′}} {σ}^{′} d {ɛ}^{′}$$

### Example 1

### Stoichiometry of Complexes

Proton NMR spectroscopy (Bruker Advance 600 MHz spectrometer) was used to measure the ratio of PSS to PDADMAC in the Polyelectrolyte complexes as follows: excess solution was removed from a piece of complex (50-100 mg) using paper wipes. To exchange most of the hydration H₂O with D₂O the complex was rinsed with 1.0 M NaCl in D₂O (in three 1 mL aliquots over 24). The piece of complex was then dissolved in 1 mL 2.5 M KBr in D₂O. For calibration, spectra of mixtures of known amounts of PSS and PDADMAC in 2.5 M KBr were recorded under the same conditions. Then the precipitates were redissolved in 2.5 M KBr in D₂O. In the solution 1H NMR spectra of these dissolved complexes, all the protons from the constituent polyelectrolytes were present. Integration of the signal of the four aromatic hydrogens of PSS (between 5.5 and 9 ppm) provided a convenient internal standard for comparison with the 16 aliphatic 1H (between 0 and 4.6 ppm) on PDADMA plus the three aliphatic 1H on PSS. The ratio PSS:PDADMA charged polyelectrolyte repeat units was 1:1, within an experimental error of +/−2%.

### Example 2

### Polyelectrolyte Complex Morphology

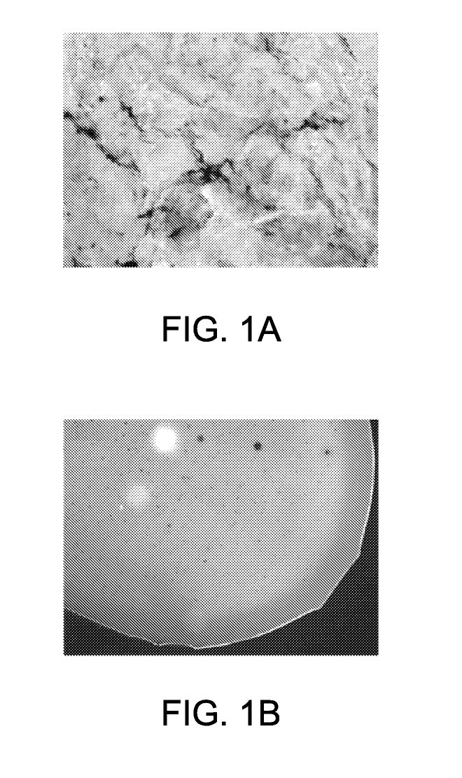

For imaging, samples soaked in DI water were cut into 10 μm slices using a cryostat microtome (Leica CM 1850) and imaged with a Nikon Eclipse Ti inverted microscope using a Photometrics Cool Snap HQ2 camera and NIS Elements AR 3.0 software. The magnification was 100× or 200×. FIGS. 1A and 1B are optical autofluorescence microscopy images of 10 μm thick slices of polyelectrolyte complexes precipitated in 0.25 M NaCl, and centrifuged. FIG. 1A depicts the polyelectrolyte complex as extruded, and FIG. 1B depicts the polyelectrolyte complex soaked in DI water. 450-490 nm excitation and 500-550 nm emission filter cube. Scale bar: 100 μm.

The autofluorescence of PSS was exploited using excitation at 485-505 nm and emission at 510-540 nm. Before extrusion, the as-precipitated polyelectrolyte complex of PSS and PDADMA was porous with the consistency of cottage cheese. See FIG. 1A. After extrusion, the pores were almost eliminated and the material was much tougher. See FIG. 1B.

### Example 3

### Polyelectrolyte Complex Water Content

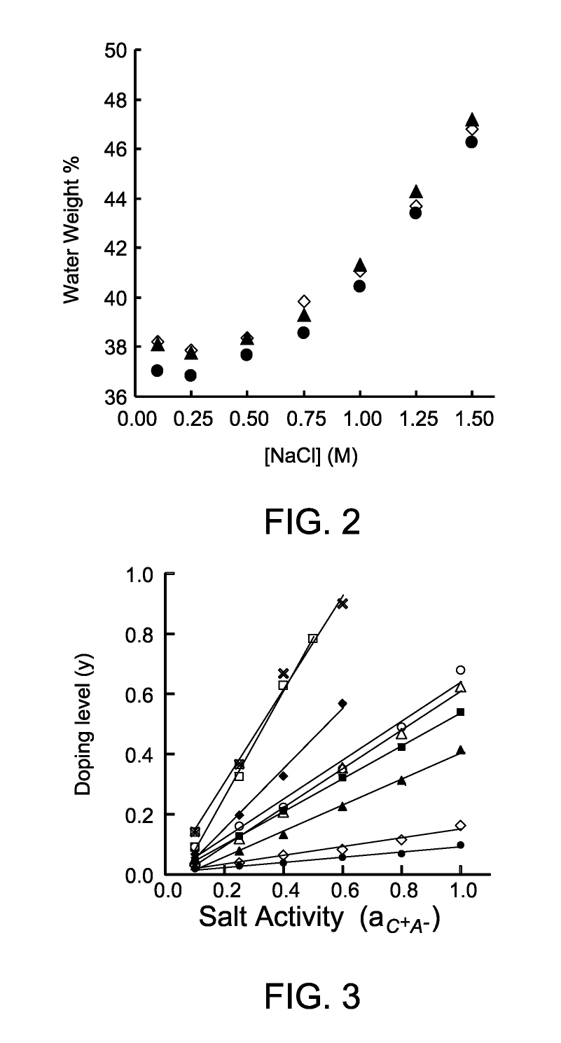

Starting polyelectrolyte complex was subjected to one, two, or three extrusions, all performed with 1M NaCl. Following extrusion, the doping level was set by immersing the article in a salt solution of specific NaCl concentration for 2 days. See FIG. 2, which is a graph depicting room temperature water content vs. salt concentration for PSS/PDADMA Polyelectrolyte complexes after hydration for 2 days in salt solutions. The data is shown for polyelectrolyte complex extruded (), double extruded (⋄), and triple extruded (▴). Excess salt solution was wiped off the articles and weighed. The samples were dried in an oven at 90° C. for 4 h and reweighed. The weight loss was the water content in weight %

### Example 4

### Doping of PSS/PDADMA with Different Salts

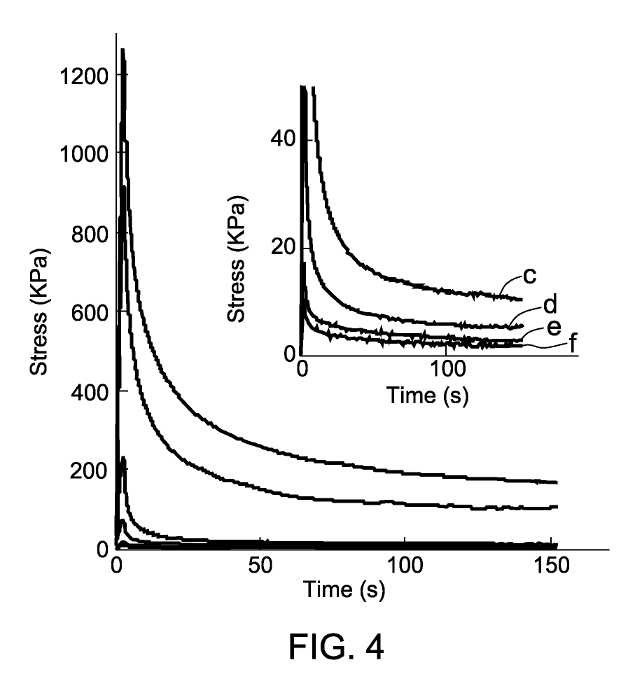

A conductivity meter, equipped with a water jacket and temperature controlled to 25° C.±0.1° C., was standardized with NaCl solutions. After two consecutive extrusions, the stoichiometric (1:1 PSS:PDADMA) extruded polyelectrolyte complex, exPECs, from the Example above were annealed in 1.5 M NaCl for 24 h, then soaked in excess water to remove all ions. The exPEC rods were cut into samples approximately 1 cm long, dabbed dry with a paper wipe and immersed separately into solutions of various salts at different concentrations. Each sample was allowed to dope to equilibrium at room temperature (23° C.±2° C.) for at least 24 h. Polyelectrolyte complexes were wiped then dropped into 50 mL water in the conductivity cell equipped with a small stir bar. Conductivity values were recorded every 30 seconds for 90 min and sent to a computer. After release of salt, exPECs were dried at 110° C. for 6 h to obtain the dry mass of the complex. All salt released was assumed to be doping the polymer. See FIG. 3, which is a graph depicting doping level, y, in PSS/PDADMA extruded polyelectrolyte complex (exPEC) versus salt activity for NaF (); NaCH₃COO (⋄); NaClO₃ (▴); NaCl (▪); NaNO₃ (Δ) NaBr (∘); NaI (♦); NaClO₄ (x); and NaSCN (□). Room temperature. FIG. 3 shows doping level as a function of salt concentration in the doping solution.

This method is reliable for doping levels up to about 0.3 only. At doping levels higher than about 0.3 additional salt not paired with charged polyelectrolyte repeat units enters the complex. Hence, doping levels higher than 0.3 in FIG. 3 are only approximate.

### Example 5

### Stress and Strain Relaxation

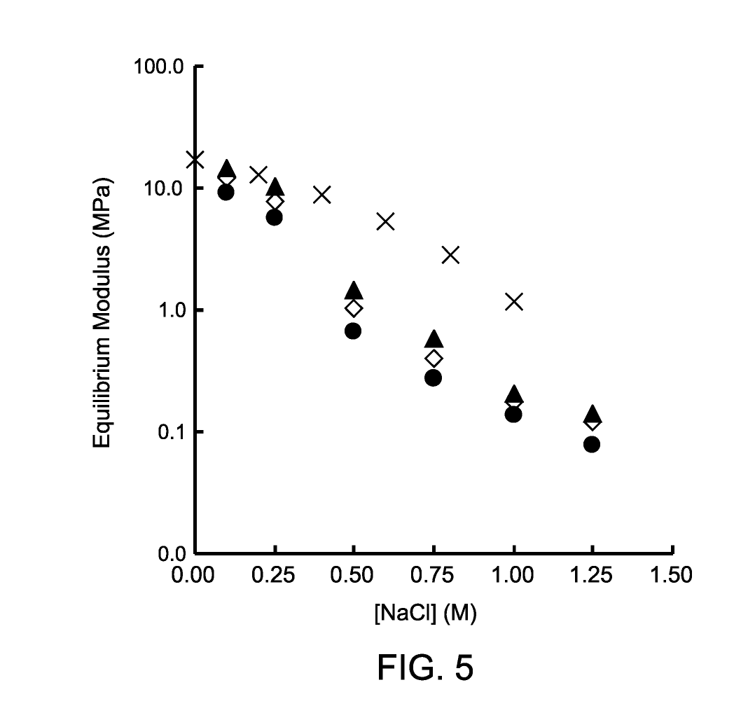

Extruded fibers of PSS/PDADMA were prepared as in the Example above. Fibers were mounted in the tensile tester, bathed in salt solution of a specific concentration, and strained rapidly to 2%. Stress at this fixed strain was measured as a function of time. The viscous component of the viscoelastic response was allowed to relax for 150 s to achieve equilibrium or steady-state stress. The equilibrium modulus is the equilibrium stress divided by the strain. Strain relaxation experiments followed the same trend: stress was removed from all strained samples which samples relaxed back to the original unstrained dimensions within a few minutes following the removal of stress. That is, no strain was stored in the material. In the present invention, strain release occurs on a stimulus whereas strain relaxation occurs spontaneously on removal of stress. See FIG. 4, which is a graph depicting stress relaxation of extruded PEC doped in different NaCl concentrations and strained rapidly to 2%: 0.1 M (a), 0.25 M (b), 0.5 M (c), 0.75 M (d), 1.0 M (e), and 1.25 M (f) NaCl.

### Example 6

### Equilibrium Modulus

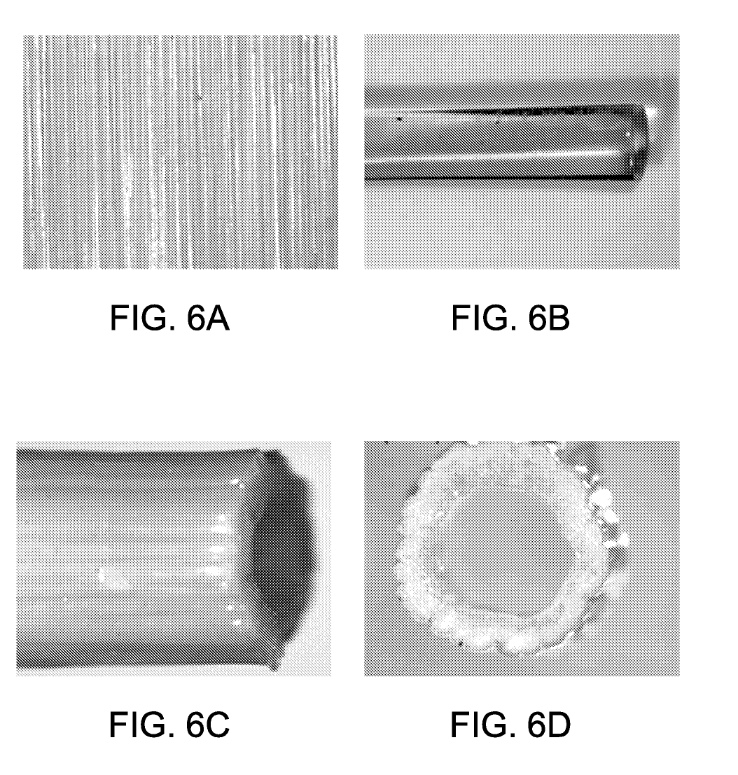

The relationship between applied strain and resulting stress in polyelectrolyte complex for strains of <2% (i.e., percent of elongation less than 2% of length of polyelectrolyte complex at rest) was found to be linear. Further, when the elongation cycle was repeated at a certain ionic strength, with a strain of less than 2%, stress/strain behavior was reproducible with minimal hysteresis. This means that the polyelectrolyte complex recovered almost completely when the applied stress is removed (i.e. there was no residual deformation). These measurements covered a range of salt concentrations. There is no evidence that strain is stored when the stress was removed. See FIG. 5, which is a graph depicting equilibrium modulus at different salt solutions for PSS/PDADMA samples extruded (), double extruded (⋄), and triple extruded (▴) at strain of 2% and speed of 10 mm/min. The points (x) are the modulus for PEMU of PDADMA/PSS recorded by Jaber et al.

### Example 7

### Extrusion of Different Shapes

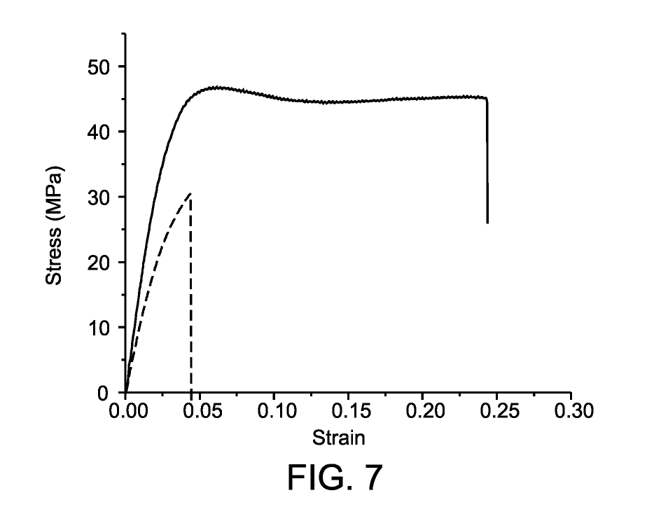

Starting polyelectrolyte was equilibrated with 1M NaCl and extruded as in the example above. The exit orifice had the geometry of tape, rod, and tube. See FIGS. 6A through 6D, which are images of extruded polyelectrolyte complex with salt. Images of an extruded polyelectrolyte complex tape (FIG. 6A), an extruded polyelectrolyte complex rod (FIG. 6B), extruded polyelectrolyte complex tube (FIG. 6C) and its cross-section (FIG. 6D). Scale bar is 0.5 mm

### Example 8

### Extrusion of Stored Strain Polyelectrolyte Complex Using Undoped Starting Polyelectrolyte Complex

The polyelectrolyte complex rod was prepared as described in the Example above. The polyelectrolyte complex rod was chopped into pieces between 5 mm and 10 mm, then soaked in water by changing the water several times until all the NaCl in the polyelectrolyte complex rod was completely removed. The fully hydrated polyelectrolyte complex rod was introduced into the hopper of the extruder with a round exit nozzle of diameter 1 mm. The extruder parameters were set as follows: rotor temperature, 98° C.; header temperature, 102° C.; gap space, 3.8 mm; and rotor speed 60% (110 rpm). The extruded complex was continuously collected on a takeup. These parameters allowed the extrusion of 1 mm fiber at approximately 2 g min⁻¹.

### Example 9

### Superior Strength of Stored Strain Polyelectrolyte Complex

Strain to break measurements were carried out on stored strain and annealed samples. The annealed samples were prepared by relaxing the stored strain complex with 1.5 M NaCl_{aq} for 24 hr, followed by removing the NaCl with water and drying at room temperature. Samples of diameter 1 mm and length 20 mm were stretched at a speed of 10 mm min⁻¹. The stored strain polyelectrolyte complex shows relative higher Young's modulus (1500±200 MPa) than the annealed polyelectrolyte complex (1100±40 MPa).

### Example 10

### Superior Toughness of Stored Strain Polyelectrolyte Complex

1 mm diameter stored strain polyelectrolyte complex was produced by the extrusion method above. In some of this material, the strain was released using 1.5M NaCl as a stimulus. The toughness was calculated by integrating the area under the stress-strain (to failure) curve (εf′).

$$Toughness = {\int }_{0}^{{ɛ}_{f}^{′}} {σ}^{′} d {ɛ}^{′}$$

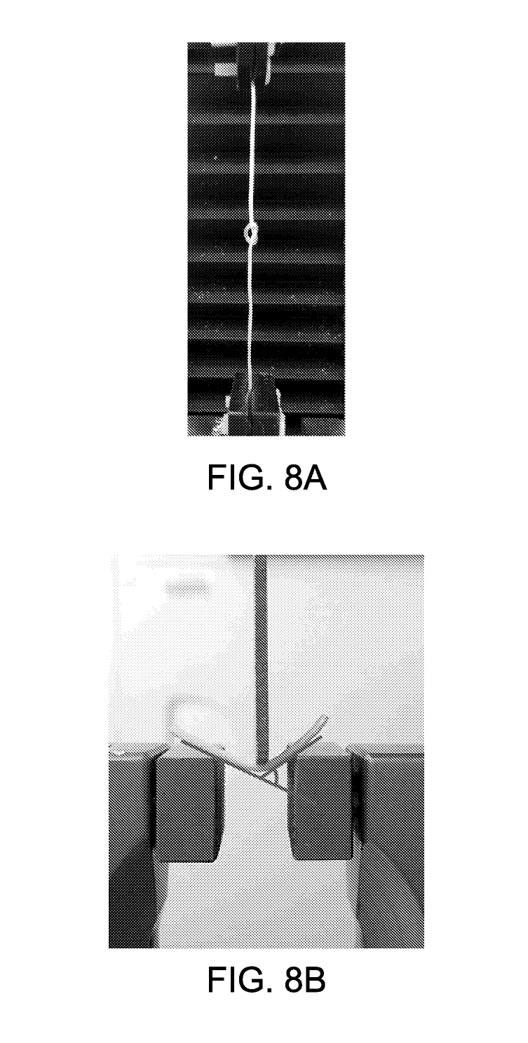

See FIG. 7, which is a graph depicting Strain to Break test for stored strain and annealed PEC fibers. Stretching speed: 10 mm min⁻¹ (50% strain-min⁻¹).

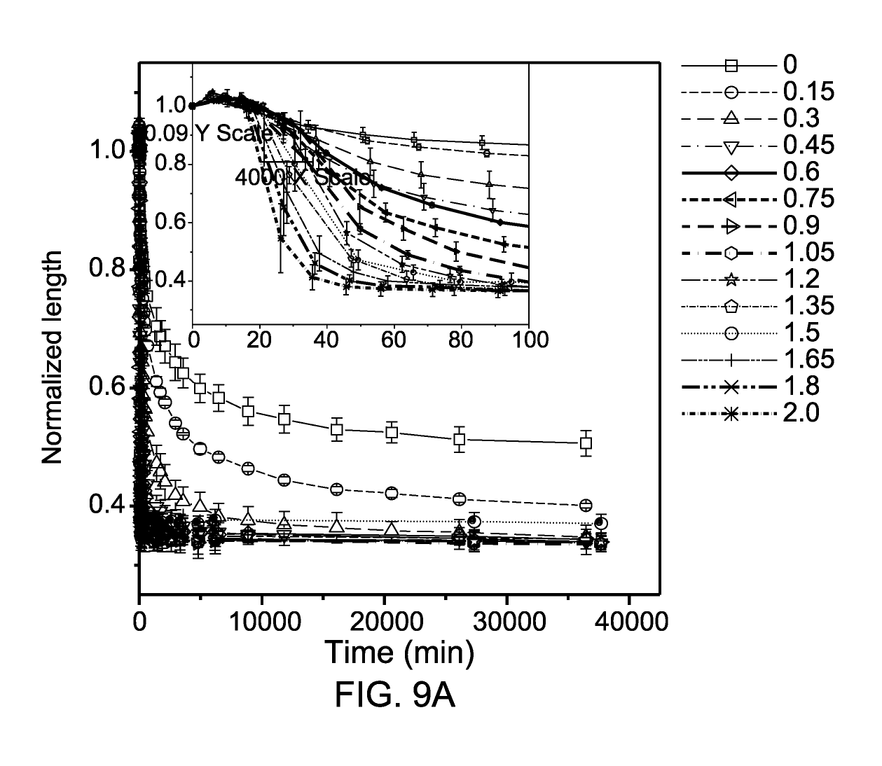

The stored strain samples show much higher toughness (9.1±2.0 MJ m⁻³) than the stimulus-relaxed unstrained samples (0.7±0.2 MJ m⁻³). In another experiment stored strain fibers could be tied in a knot without breaking, whereas fibers where the strain had been released with a stimulus (soaking in 1.5 M NaCl) were much more fragile and could only be bent to 58°, as shown in FIGS. 8A and 8B, which are photographs of stored strain fibers in a tight knot (FIG. 8A) and the maximum degree (˜58°) the annealed sample can be bent (FIG. 8b).

### Example 11

### Release of Stored Strain by Salt Solution Stimulus

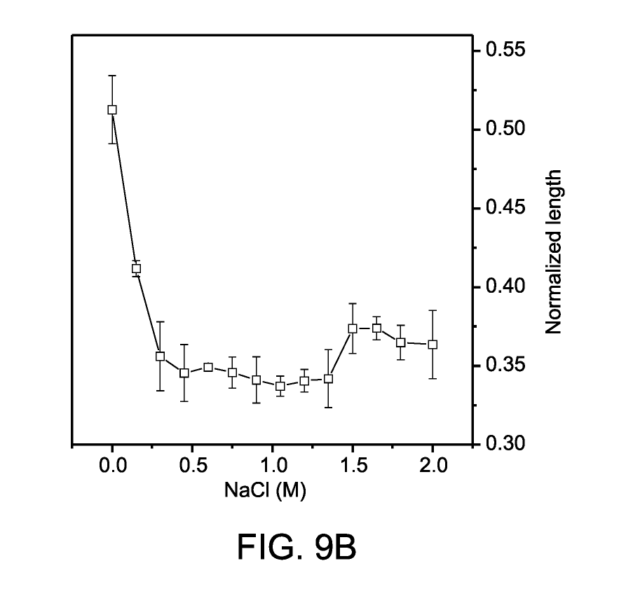

To measure dimensional changes in stored strain extruded polyelectrolyte complex on stimulus in NaCl_{aq}, samples were soaked in solutions of different [NaCl] and imaged continuously. The length of the PEC fibers at time t was divided by the original length (before stimulus). For the first 20 min the all the polyelectrolyte complexes swelled slightly due to hydration. Thereafter, the stored strain started to release. The higher concentration of the NaCl stimulus solution the faster the polyelectrolyte complexes shortened. For [NaCl] higher than 0.9 M, polyelectrolyte complexes can reach their minimum length (maximum contraction) in about 2 hr. As shown in FIGS. 9A and 9B, normalize length is the reciprocal of the strain ratio. FIGS. 9A and 9B depict length change (contraction) of stored strain polyelectrolyte complex samples in NaCl solutions (0-2.0 M) with time (FIG. 9A). And the minimum length in solutions of different [NaCl] (FIG. 9B). The stored strain ratio is about 3 for salt concentrations 0.25M and higher. It is seen that hydration by itself (immersion in salt-free water) is not enough to relieve all the stored strain whereas the salt concentrations greater than 0.3M released the maximum strain. The strain ratio for water by itself was less than 2 and for NaCl solutions >0.3M the ratio was between 2.5 and 3.

### Example 12

### Release of Stored Strain by Hot Water

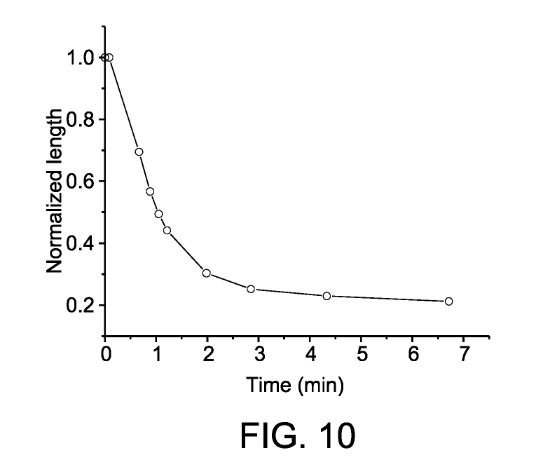

To measure dimensional changes during annealing of as-extruded salt free stored strain polyelectrolyte in hot water, samples were soaked in hot water (about 90° C.) and imaged continuously. For the first 5 to 10 seconds, the PEC was hydrated by hot water. Then the PEC contracted quickly. 95% of the contraction can be accomplished in less than 3 min. The equilibrium length was 21.2% of the original length. See FIG. 10, which is a graph depicting release of stored strain polyelectrolyte complex by 90° C. water. The stored strain ratio is about 5.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and processes without departing from the scope of the invention, it is intended that all matter contained in the above description and shown in the accompanying drawings shall be interpreted as illustrative and not in a limiting sense.

What is claimed is:

1. An article comprising a polyelectrolyte complex comprising an interpenetrating network of at least one predominantly positively charged polyelectrolyte polymer and at least one predominantly negatively charged polyelectrolyte polymer, the polyelectrolyte complex further comprising stored strain with a stored strain factor of at least 2.

2. The article of claim 1 wherein the polyelectrolyte complex is at least about 10 micrometers thick.

3. The article of claim 1 wherein the salt doping level is less than about 0.1.

4. The article of claim 1 wherein the salt doping level is less than about 0.05.

5. The article of claim 1 wherein the salt doping level is less than about 0.01.

6. The article of claim 1 wherein the polyelectrolyte complex comprises pores in a pore volume between about 10% and about 90% of the total volume of the article.

7. The article of claim 1 wherein the polyelectrolyte complex comprises pores in a pore volume less than about 1% of the total volume of the article.

8. The article of claim 1 wherein the polyelectrolyte complex comprises pores in a pore volume less than about 0.1% of the total volume of the article.

9. The article of claim 1 wherein the polyelectrolyte complex has a Young's modulus of at least about 2000 MPa.

10. The article of claim 1 wherein the polyelectrolyte complex has a toughness of at least about 2 MJ m⁻³.

11. The article of claim 1 wherein the polyelectrolyte complex comprises crosslinking at a level of chemical crosslinking between about 0.01% and about 50% as measured as a percentage of total ion pairs within the polyelectrolyte complex.

12. The article of claim 1 wherein the polyelectrolyte complex further comprises one or more additives selected from the group consisting of metal oxide particles, silicon oxide, zirconium oxide, inorganic minerals, clay minerals, carbon powder, graphite, carbon fibers, carbon nanotubes, polymer fibers, cellulose fibers, metal particles, metal fibers, magnetic particles and combinations thereof.

13. The article of claim 1 further comprising a pharmaceutical agent.

14. The article of claim 1 further comprising bioadhesive.

15. The article of claim 1 further comprising chemical crosslinks

16. The article of claim 1 further comprising zwitterionic or oxoethylene functionality.

17. The article of claim 1 wherein the polyelectrolyte complex further comprises an additive selected from the group consisting of an antibacterial agent, an anti-viral agent, an anti-inflammation agent, an anti-rejection agent, a growth factor, a growth hormone, and any combination thereof.

18. A method of releasing stored strain from the article of claim 1, the method comprising:
contacting the polyelectrolyte complex having stored strain with water to thereby hydrate the polyelectrolyte complex; and
exposing the hydrated polyelectrolyte complex to a stimulus sufficient to release stored strain from the polyelectrolyte complex, said stimulus being selected from the group consisting of salt concentration increase, temperature increase, and pH change.

19. A method of forming an article comprising a polyelectrolyte complex comprising an interpenetrating network of at least one predominantly positively charged polyelectrolyte polymer and at least one predominantly negatively charged polyelectrolyte polymer, the polyelectrolyte complex further comprising stored strain with a stored strain factor of at least 2, the method comprising:
contacting a substantially undoped polyelectrolyte complex comprising an interpenetrating network of at least one predominantly positively charged polyelectrolyte polymer and at least one predominantly negatively charged polyelectrolyte polymer with water to thereby hydrate the polyelectrolyte complex; and
applying an external stress to the hydrated polyelectrolyte complex, the external stress sufficient to increase at least one dimension of the hydrated polyelectrolyte complex.

20. The method of claim 19 wherein the salt doping level of the polyelectrolyte complex is less than about 0.1.

21. The method of claim 19 wherein the salt doping level of the polyelectrolyte complex is less than about 0.05.

22. The method of claim 19 wherein the salt doping level of the polyelectrolyte complex is less than about 0.01.

23. The method of the claim 19 wherein the external stress comprises a mechanical force.

24. The method of claim 23 wherein the mechanical force comprises extrusion through an orifice.
